SEQUENCE LISTING (83 sequences)

SEQ ID NO: 1 - protein, 7 aa, Artificial Sequence
  Lyn Position 5 is D-lysine
IVTYKKI

SEQ ID NO: 2 - protein, 8 aa, Artificial Sequence
  Lyn
GIVTYGKI

SEQ ID NO: 3 - protein, 11 aa, Artificial Sequence
  Lyn
GIVTYGKIPYP

SEQ ID NO: 4 - protein, 6 aa, Artificial Sequence
  Lyn
GIVTYG

SEQ ID NO: 5 - protein, 9 aa, Artificial Sequence
  Lyn
GLVTYKKIK

SEQ ID NO: 6 - protein, 8 aa, Artificial Sequence
  Lyn
GLVTYGKI

SEQ ID NO: 7 - protein, 8 aa, Artificial Sequence
  Lyn
GIVSYGKI

SEQ ID NO: 8 - protein, 8 aa, Artificial Sequence
  Lyn
GIVTYAKI

SEQ ID NO: 9 - protein, 8 aa, Artificial Sequence
  Lyn
GIVTFGKI

SEQ ID NO: 10 - protein, 8 aa, Artificial Sequence
  Lyn
GIVTYGKV

SEQ ID NO: 11 - protein, 8 aa, Artificial Sequence
  Lyn
GIVTYGXI

SEQ ID NO: 12 - protein, 8 aa, Artificial Sequence
  Lyn
GIVSYGXI

SEQ ID NO: 13 - protein, 8 aa, Artificial Sequence
  Lyn
GVVTYGKI

SEQ ID NO: 14 - protein, 8 aa, Artificial Sequence
  Lyn
GIATYGKI

SEQ ID NO: 15 - protein, 8 aa, Artificial Sequence
  Lyn
GIITYGKI

SEQ ID NO: 16 - protein, 8 aa, Artificial Sequence
  Lyn
GILTYGKI

SEQ ID NO: 17 - protein, 8 aa, Artificial Sequence
  Lyn
GLVTYDKI

SEQ ID NO: 18 - protein, 8 aa, Artificial Sequence
  Lyn
GLVTYKKI

SEQ ID NO: 19 - protein, 8 aa, Artificial Sequence
  Lyn
GLVTYSKI

SEQ ID NO: 20 - protein, 8 aa, Artificial Sequence
  Lyn
GLVTYMKI

SEQ ID NO: 21 - protein, 6 aa, Artificial Sequence
  Lyn
GIVTYK

SEQ ID NO: 22 - protein, 8 aa, Artificial Sequence
  Lyn
GIVTYKKI

SEQ ID NO: 23 - protein, 8 aa, Artificial Sequence
  Lyn
GLVTYKKI

SEQ ID NO: 24 - protein, 8 aa, Artificial Sequence
  Lyn
GIVTYGKI

SEQ ID NO: 25 - protein, 8 aa, Artificial Sequence
  Lyn
GLVTYKKI

SEQ ID NO: 26 - protein, 8 aa, Artificial Sequence
  Lyn Position 5 is di-iodo tyrosine
GLVTYKKI

SEQ ID NO: 27 - protein, 8 aa, Artificial Sequence
  Lyn
GIVTYGKI

SEQ ID NO: 28 - protein, 8 aa, Artificial Sequence
  Lyn
GLVTYKKI

SEQ ID NO: 29 - protein, 8 aa, Artificial Sequence
  Lyn Position 5 is di-iodo tyrosine
GLVTYKKI

SEQ ID NO: 30 - protein, 8 aa, Artificial Sequence
  Lyn
GLVTYKKI

SEQ ID NO: 31 - protein, 9 aa, Artificial Sequence
  Lyn Position 5 is di-iodo tyrosine
GLVTYKKIK

SEQ ID NO: 32 - protein, 9 aa, Artificial Sequence
  Lyn Position 5 is di-iodo tyrosine
GLVTYKKIK

SEQ ID NO: 33 - protein, 9 aa, Artificial Sequence
  Lyn Position 5 is di-iodo tyrosine
GLVTYKKIK

SEQ ID NO: 34 - protein, 11 aa, Artificial Sequence
  Lyn Position 6 is D-isomer of lysine
GLVTYKKIPYP

SEQ ID NO: 35 - protein, 11 aa, Artificial Sequence
  Lyn Position 5 is di-iodo tyrosine
GLVTYKKIPYP

SEQ ID NO: 36 - protein, 8 aa, Artificial Sequence
  Lyn Position 5 is di-iodo tyrosine
GIATYKKI

SEQ ID NO: 37 - protein, 8 aa, Artificial Sequence
  Lyn Position 5 is di-iodo tyrosine
GLVTYDKI

SEQ ID NO: 38 - protein, 9 aa, Artificial Sequence
  Lyn Position 6 is D-isomer of lysine
GIATYKKIK

SEQ ID NO: 39 - protein, 9 aa, Artificial Sequence
  Lyn Position 6 is D-isomer of lysine
GLVTYKKIK

SEQ ID NO: 40 - protein, 9 aa, Artificial Sequence
  Lyn Position 1 is oleoyl-glycine Position 6 is D-isomer of lysine
GIATYKKIK

SEQ ID NO: 41 - protein, 9 aa, Artificial Sequence
  Lyn Position 1 is oleoyl-glycine Position 6 is D-isomer of lysine
GLVTYKKIK

SEQ ID NO: 42 - protein, 9 aa, Artificial Sequence
  Lyn Position 6 is D-isomer of lysine
GLVTYKKIK

SEQ ID NO: 43 - protein, 9 aa, Artificial Sequence
  Lyn Position 1 is stearyl-glycine Position 6 is D-isomer of lysine
GLVTYKKIK

SEQ ID NO: 44 - protein, 9 aa, Artificial Sequence
  Lyn Position 1 is phenyl-glycine Position 6 is D-isomer of lysine
GLVTYKKIK

SEQ ID NO: 45 - protein, 9 aa, Artificial Sequence
  Lyn Position 1 is adamantan- glycine Position 6 is D-isomer of lysine
GLVTYKKIK

SEQ ID NO: 46 - protein, 9 aa, Artificial Sequence
  Lyn Position 1 is naphthalene- glycine Position 6 is D-isomer of lysine
GLVTYKKIK

SEQ ID NO: 47 - protein, 9 aa, Artificial Sequence
  Lyn Position 1 is myristoleyl- glycine Position 6 is D-isomer of lysine
GLVTYKKIK

SEQ ID NO: 48 - protein, 9 aa, Artificial Sequence
  Lyn Position 1 is toluen-glycine Position 6 is D-isomer of lysine
GLVTYKKIK

SEQ ID NO: 49 - protein, 9 aa, Artificial Sequence
  Lyn Position 1 is biphenyl-glycine Position 6 is D-isomer of lysine
GLVTYKKIK

SEQ ID NO: 50 - protein, 9 aa, Artificial Sequence
  Lyn Position 1 is Cinnamoyl-glycine Position 6 is D-isomer of lysine
GLVTYKKIK

SEQ ID NO: 51 - protein, 9 aa, Artificial Sequence
  Lyn Position 1 is nitrobenzoyl- glycine Position 6 is D-isomer of lysine
GLVTYKKIK

SEQ ID NO: 52 - protein, 9 aa, Artificial Sequence
  Lyn Position 1 is toluoyl-glycine Position 6 is D-isomer of lysine
GLVTYKKIK

SEQ ID NO: 53 - protein, 9 aa, Artificial Sequence
  Lyn Position 1 is furoyl-glycine Position 6 is D-isomer of lysine
GLVTYKKIK

SEQ ID NO: 54 - protein, 9 aa, Artificial Sequence
  Lyn Position 1 is benzoyl-glycine Position 6 is D-isomer of lysine
GLVTYKKIK

SEQ ID NO: 55 - protein, 9 aa, Artificial Sequence
  Lyn Position 1 is oleoyl-glycine Position 6 is D-isomer of lysine
GLVTYKKIK

SEQ ID NO: 56 - protein, 9 aa, Artificial Sequence
  Lyn Position 1 is cyclohexane- glycine Position 6 is D-isomer of lysine
GLVTYKKIK

SEQ ID NO: 57 - protein, 9 aa, Artificial Sequence
  Lyn Position 1 is norbornane- glycine Position 6 is D-isomer of lysine
GLVTYKKIK

SEQ ID NO: 58 - protein, 9 aa, Artificial Sequence
  Lyn Position 1 is z-caproic-glycine Position 6 is D-isomer of lysine
GLVTYKKIK

SEQ ID NO: 59 - protein, 9 aa, Artificial Sequence
  Lyn Position 1 is oleyl-glycine Position 6 is D-isomer of lysine
GLVTYKKIK

SEQ ID NO: 60 - protein, 9 aa, Artificial Sequence
  Lyn Position 6 is D-isomer of lysine
GLVTYKKIK

SEQ ID NO: 61 - protein, 9 aa, Artificial Sequence
  Lyn Position 5 is di-iodo tyrosine
GLVTYKKIK

SEQ ID NO: 62 - protein, 9 aa, Artificial Sequence
  Lyn Position 1 is oleyl-glycine Position 6 is D-isomer of lysine
GLVTYKKIK

SEQ ID NO: 63 - protein, 9 aa, Artificial Sequence
  Lyn Position 1 is ricinolelyl-glycine Position 6 is D-isomer of lysine
GLVTYKKIK

SEQ ID NO: 64 - protein, 9 aa, Artificial Sequence
  Lyn Position 6 is D-isomer of lysine
GLVTYKKIK

SEQ ID NO: 65 - protein, 9 aa, Artificial Sequence
  Lyn Position 5 is di-iodo tyrosine
GLVTYKKIK

SEQ ID NO: 66 - protein, 8 aa, Artificial Sequence
  Lyn Position 1 is palmitoyl-glycine
GIVTYGKI

SEQ ID NO: 67 - protein, 8 aa, Artificial Sequence
  Lyn Position 1 is palmitoyl-glycine
GIVSYGXI

SEQ ID NO: 68 - protein, 8 aa, Artificial Sequence
  Lyn Position 1 is stearyl-glycine
GIVTYGKI

SEQ ID NO: 69 - protein, 8 aa, Artificial Sequence
  Lyn Position 1 is stearyl-glycine
GIITYGKI

SEQ ID NO: 70 - protein, 8 aa, Artificial Sequence
  Lyn Position 1 is stearyl-glycine
GIFTYGKI

SEQ ID NO: 71 - protein, 8 aa, Artificial Sequence
  Lyn Position 1 is stearyl-glycine
GILTYGKI

SEQ ID NO: 72 - protein, 6 aa, Artificial Sequence
  Lyn Position 1 is stearyl-glycine Position 6 is D-isomer of lysine
GIVTYK

SEQ ID NO: 73 - protein, 8 aa, Artificial Sequence
  Lyn Position 1 is stearyl-glycine Position 6 is D-isomer of lysine
GLVTYKKI

SEQ ID NO: 74 - protein, 8 aa, Artificial Sequence
  Lyn Position 1 is stearyl-glycine Position 6 is D-isomer of lysine
GIVTYKKI

SEQ ID NO: 75 - protein, 9 aa, Artificial Sequence
  Merystate at position 1
GLVTYKKIK

SEQ ID NO: 76 - protein, 25 aa, Artificial Sequence
  synthetic
YEIVTYGKIPYPGRTNADVMTALSQ

SEQ ID NO: 77 - protein, 6 aa, Artificial Sequence
  synthetic
IVTYGK

SEQ ID NO: 78 - protein, 19 aa, Artificial Sequence
  synthetic
ITEYMAKGSLLDFLKSDEG

SEQ ID NO: 79 - protein, 7 aa, Artificial Sequence
  synthetic
SLLDFLK

SEQ ID NO: 80 - protein, 9 aa, Artificial Sequence
  synthetic
LYAVVTREE

SEQ ID NO: 81 - protein, 18 aa, Artificial Sequence
  synthetic
ANLMKTLQHDKLVRLYAV

SEQ ID NO: 82 - DNA, 30 bp, Artificial Sequence
  Antisense
atgggatgtataaaatcaaaagggaaagac

SEQ ID NO: 83 - protein, 7 aa, Artificial Sequence
  synthetic
KGYTVIG

(19) United States
(12) Patent Application Publication
(10) Pub. No.: US 2002/0151497 A1
(43) Pub. Date: Oct. 17, 2002

(54) Treatment of prostate cancer by inhibiting Lyn tyrosine kinase

(75) Inventor: Shmuel Ben-Sasson, Jerusalem (IL)

Correspondence Address: BROWDY AND NEIMARK, P.L.L.C., 624 NINTH STREET, NW, SUITE 300, WASHINGTON, DC, 20001-5303, US

(73) Assignee: Children's Medical Center Corporation, Boston, MA (US)

(21) Appl. No.: 10/012,030

(22) Filed: Dec. 11, 2001

Related U.S. Application Data
(63) Continuation-in-part of application No. 09/735,279, filed on Dec. 11, 2000.

(51) Int. Cl. A61K 38/10; A61K 38/08

(52) U.S. Cl. 514/12; 514/13; 514/14; 514/15; 514/16; 514/17

(57) ABSTRACT

The present invention concerns methods for the treatment of prostate cancer by the inhibition of Lyn-kinase associated signal transduction. Preferred in accordance with the invention are inhibitors which comprise sequences derived from specific regions of the Lyn-kinase.

### CROSS-REFERENCE TO RELATED APPLICATIONS

This application is a continuation-in-part of U.S. application Ser. No. 09/735,279, filed on Dec. 11, 2000, the content of which is incorporated herein entirely by reference.

### BACKGROUND OF THE INVENTION

Protein tyrosine kinases are members of the eukaryotic protein kinase superfamily. Enzymes of this class specifically phosphorylate tyrosine residues of intracellular proteins and are important in mediating signal transduction in multicellular organisms. Protein tyrosine kinases occur as membrane-bound receptors, which participate in transmembrane signaling, or as intracellular proteins which take part in signal transduction within the cell, including signal transduction to the nucleus.

As such, phosphorylation of tyrosine by protein tyrosine kinases is an important mechanism for regulating intracellular events in response to environmental changes. A wide variety of cellular events, including cytokine responses, antigen-dependent immune responses, cellular transformation by RNA viruses, oncogenesis, regulation of the cell cycle and modification of cell morphology and phenotype are regulated by protein tyrosine kinases.

Enhanced protein tyrosine kinase activity can lead to persistent stimulation by secreted growth factors, for example, which, in turn, can lead to proliferative diseases such as cancer, to nonmalignant proliferative disease such as arteriosclerosis, psoriasis and to inflammatory response such as septic shock.

Src is among the first protein kinases described whose uncontrolled expression is directly linked to malignant transformation. The Src family contains several members. Some of these members are ubiquitously expressed while others like Lck, Hck, and Lyn have been, until recently, thought to be expressed primarily in cells of the immune system.

Prostate cancer is a malignancy with high incidence in many countries. It is also a cancer with high morbidity and mortality rates and constitutes one of the leading causes of death or disability among males. Its early detection and eradication is aggressively pursued worldwide.

There exists a need for further methods to alleviate and eliminate prostate cancer. In particular, there exists a need for substances which can be administered to individuals who have or are susceptible to developing prostate cancer

### SUMMARY OF THE INVENTION

The present invention is based on the surprising finding that short peptides, corresponding to short sequences from specific regions of Lyn-kinase, or variants of said sequences, were able to reduce growth of prostate cancer cells both in vivo and in vitro.

The present invention is further based on the surprising finding that said short peptides were capable of inhibiting Lyn-associated signal transduction, thus leading to the understanding that the inhibition of the Lyn-associated signal transduction (hereinafter “LAST”) leads to the reduction of growth of prostate cancer cells.

Thus, by a first aspect, the present invention concerns a method for the reduction of growth of prostate cancer cells comprising: administering to the cells a compound comprising an amino acid sequence that corresponds to sequences in specific regions of Lyn-kinase (hereinafter: the “HJ-loop, B4-B5 region, αD region, A-region”) or to variants of said sequence.

The method of reduction of growth of prostate cancer cells can be used as a therapeutic method for the treatment of prostate cancer.

The present invention further concerns methods for identifying the variants of said sequences effective in the reduction of growth of prostate cancer cells.

By a second aspect, the present invention concerns a method for reducing growth of prostate cancer cells by administration to the cells of at least one inhibitor of LAST.

The method may be used as a therapeutic method for the treatment of prostate cancer.

The inhibitors of LAST may be compounds comprising amino acid sequences corresponding to sequences present in the above specific regions of the Lyn- kinase, or variants of said sequences; antisense sequences corresponding to a portion of the Lyn-kinase gene or Lyn-kinase mRNA, so that hybridization between the two can reduce protein expression; dominant negative Lyn-kinases; ribozymes capable of specifically cleaving Lyn-kinase RNA; and small organic molecules capable of inhibiting LAST.

The most preferred inhibitors of the LAST, in accordance with the present invention, are compounds which comprise short amino acid sequences corresponding to sequences present in the above specific regions of a Lyn-kinase, or variants of said sequence. More preferably the region is the HJ-loop.

Without wishing to be bound by theory, it is assumed that the amino acid sequence, present in said compounds, mimics these specific regions in the Lyn-kinase, which are regions that interact with other cellular components, , such as with the substrates of the Lyn-kinase, phosphatases of the kinase, or other kinases that de-phosphorylate, or phosphorylate, respectively, the Lyn-kinase. This mimic sequence is assumed to bind to the other cellular components (for example to the substrates of the Lyn-kinase) and this binding causes the interruption of the interaction of the Lyn-kinase with said cellular components. This interruption causes the inhibition of the signal transduction mediated by the Lyn-kinase, thus leading to the reduction of the growth of prostrate cancer cells.

### GENERAL DESCRIPTION OF THE INVENTION

By one aspect, the present invention concerns a method for the reduction in the growth of prostate cancer cells the method comprising:

contacting the cells with an effective amount of a compound comprising a sequence selected from:

(a) a sequence which is a continuous stretch of at least five amino acids present in Lyn-kinase in positions 434-458 (HJ loop);

(b) a sequence which is a continuous stretch of at least five amino acids present in Lyn kinase in positions 318-336 (αD region);

(c) a sequence which is a continuous stretch of at least five amino acids present in Lyn-kinase in positions 305-316 (B4-B5 region);

(d) a sequence which is a continuous stretch of at least five amino acids present in a native Lyn kinase in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant, and provided that the variant maintains the biological activity of the parent sequences of (a) to (d);

(f) a sequence of any one of (a) to (e) wherein at least one of the amino acids is replaced by the corresponding D- amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h).

The term “reduction of growth” refers to a decrease in at least one of the following: number of cells (due to cell death which may be necrotic, apoptotic or a combination of the above) as compared to control; decrease in growth rates of cells, i.e. the to total number of cells may increase but at a lower level or at a lower rate than the increase in control; decrease in the invasiveness of cells (as determined for example by soft agar assay) as compared to control even if their total number has not changed; and progression of non-differentiated cancer cells to a more differentiated phenotype.

Reduction of growth in the contexts of a treated individual is a clinical term refering to at least one of: decrease in tumor size; decrease in rate of tumor growth; stasis of tumor size; decrease in the number of metastasis; decrease in the number of additional metastasis; decrease in invasiveness of the cancer; decrease in the rate of progression of the tumor from one stage to the next, as well as decrease in the angiogenesis induced by the cancer.

The term “compound (comprising sequence)” refers to a compound that includes within any of the sequences of (a) to (i) as defined above. The compound may be composed mainly from amino acid residues, and in that case the amino acid component of the compounds should comprise no more than a total of about 35 amino acids. Where the compound is mainly an amino acid compound, it may comprise of any one of the amino acid sequences of (a) to (h), a combination of two or more, preferably of three most preferably of two, of the sequences of (a) to (h) linked to each other (either directly or via a spacer moiety). The compound may further comprise any one of the amino acids sequences, or combinations as described above (in (a) to (i) above), together with additional amino acids or additional amino acid sequences. The additional amino acids may be sequences from other regions of the Lyn- kinase, for example sequences that are present in the kinase vicinity of the above regions (HJ loop, A-region, αD-region, B4-B5), N-terminal or C-terminal to the sequences of (a) to (d), or sequences which are not present in the Lyn-kinase but were included in the compound in order to improve various physiological properties such as penetration into cells (sequences which enhance penetration through membranes or barriers); decreased degradation or clearance; decreased repulsion by various cellular pumps, improved immunogenic activities, improvement in various modes of administration (such as attachment of various sequences which allow penetration through various barriers, through the gut, etc.); increased specificity, increased affinity, decreased toxicity, and the like. A specific example is the addition of the amino acid Gly, or of several Gly residues in tandem, to N-terminal of the sequence.

The compound may also comprise non-amino acid moieties, such as for example, hydrophobic moieties (various linear, branched, cyclic, polycyclic or hetrocyclic hydrocarbons and hydrocarbon derivatives) attached to the peptides of (a) to (i) to improve penetration; various protecting groups, especially where the compound is linear, which are attached to the compound's terminals to decrease degradation. Chemical (non-amino acid) groups present in the compound may be included in order to improve various physiological properties such as penetration into cells (sequences which enhance penetration through membranes or barriers); decreased degradation or clearance; decreased repulsion by various cellular pumps, improve immunogenic activities, improve various modes of administration (such as attachment of various sequences which allow penetration through various barriers, through the gut, etc.); increased specificity, increased affinity, decreased toxicity, for imaging purposes and the like. A specific example is the addition of the amino acid Gly, or of several Gly residues in tandem, to N-terminal of the sequence. The chemical groups may serve as various spacers, placed for example, between one or more of the above amino acid sequences, so as to spatially position them in suitable order in respect of each other.

The compound of the invention may be linear or cyclic, and cyclization may take place by any means known in the art. Where the compound is composed predominantly of amino acids/amino acid sequences, cyclization may N- to C-terminal, N-terminal to side chain and N-terminal to backbone, C-terminal to side chain, C-terminal to backbone, side chain to backbone and side chain to side chain, as well as backbone to backbone cyclization. Cyclization of the compound may also take place through the non-amino acid organic moieties.

The association between the amino acid sequence component of the compound and other components of the compound may be by covalent linking, by non-covalent complexion, for example, by complexion to a hydrophobic polymer, which can be degraded or cleaved producing a compound capable of sustained release; by entrapping the amino acid part of the compound in liposomes or micelles to produce the final compound of the invention. The association may be by the entrapment of the amino acid sequence within the other component (liposome, micelle) or the impregnation of the amino acid sequence within a polymer to give the final compound of the invention.

Preferably the compounds comprise an amino acid sequence of (a) to (i) above in association with (in the meaning described above) a moiety for transport across cellular membranes.

The term “moiety for transport across cellular membranes” refers to a chemical entity, or a composition of matter (comprising several entities) that causes the transport of members associated (see above) with it through phospholipdic membranes. One example of such moieties are hydrophobic moieties such as linear, branched, cyclic, polycyclic or hetrocyclic substituted or non-substituted hydrocarbons. Another example of such a moiety are short peptides that cause transport of molecules attached to them into the cell by, gradient derived, active, or facilitated transport. Other examples of other non-peptidic moieties known to be transported through membranes such as glycosylated steroid derivatives, are well known in the art. Yet another example are moieties that are endocytosed by cellular receptors such as ligands of the EGF and tranferrin receptors. The moiety of the compound may be a polymer, liposome or micelle containing, entrapping or incorporating the amino acid sequence therein. In the above examples the compound of the invention is the polymer, liposome micelle etc. impregnated with the amino acid sequence.

The term “a sequence which is a continuous stretch of at least 5 amino acids present . . . ” means any continuous stretch of having a minimum of 5 amino acids to a maximum of the full length of the region, which are present within or is an amino acid sequence described by reference to positions of Lyn -kinase. For example, in the HJ-loop defined as positions 434-458 of the Lyn-kinase, the continuous stretch of at least 5 amino acids may be from amino acid at position 434 to 438, from 435 to 439, from 436 to 440, . . . 444-458. The continuous sequence may also be of 5, 6 (435 to 440 . . . 453 to 456), 7, 8, 9, 10, 11, 12, 13, 14, 15, 16, 17, 18, 19 or 20 amino acids, obtained from each of these regions.

The term “Lyn-kinase” in reference to specific positions concerns protein tyrosine kinase denoted as EC 2.7.1.12, splice from A-human Accession TVHULY, PID g66782 (NCBI database).

The term “wherein up to 40% of amino acids of the native sequence have been replaced with a naturally or non-naturally occurring amino acid or with a peptidomimetic organic moiety” in accordance with the present invention, concerns an amino acid sequence, which shares at least 60% of its amino acid with the native sequence as described in (a), (b), (c) or (d) above, but some of the amino acids were replaced either by other naturally occurring amino acids, (both conservative and non-conservative substitutions), by non-naturally occurring amino acids (both conservative and non-conservative substitutions), or with organic moieties which serve either as true peptidomimetics (i.e. having the same steric and electrochemical properties as the replaced amino acid), or merely serve as spacers in lieu of an amino acid, so as to keep the spatial relations between the amino acid spanning this replaced amino acid. Guidelines for the determination of the replacements and substitutions are given in the detailed description part of the specification. Preferably no more than 30%, 25% or 20% of the amino acids are replaced.

The term “wherein up to 40% of the amino acids have their side chains chemically modified” refers to a variant which has the same type of amino acid residue, but to its side chain a functional group has been added. For example, the side chain may be phosphorylated, glycosylated, fatty acylated, acylated, iondiated or carboxyacylated. Other examples of chemical substitutions are known in the art and given below.

The term “up to 20% of the amino have been deleted” refer to an amino acid sequence which maintains at least 20% of its amino acid. Preferably no more than 10% of the amino acids are deleted and more preferably none of the amino acids are deleted.

The term “provided that at least 50% of the amino acids in the parent protein are maintained unaltered in the variants” the up to 40% substitution, up to 40% chemical modification and up to 20% deletions are combinatorial, i.e. the same variant may have substitutions, chemical modifications and deletions so long as at least 50% of the native amino acids are identical to those of the native sequence both as regards the amino acid and its position. In addition, the properties of the parent sequence, in modulating Lyn-associate signal transduction, have to be maintained in the variant typically, at the same or higher level.

When calculating 40% (or 35, 30, 25, 20%) replacement of 20% (or 10%) deletion from sequences, the number of actual amino acids should be rounded mathematically, so that both 40% of an 11 mer sequence (4.4) and 40% of a 12 mer sequence (4.8) is four amino acids, and only 40% of a 13 mer sequence (5.2) is five amino acids.

Typically “essential amino acids” are maintained or replaced by conservative substitutions while non-essential amino acids may be maintained, deleted or replaced by conservative or non-conservative replacements. Generally, essential amino acids are determined by various Structure-Activity-Relationship (SAR) techniques (for example amino acids when replaced by Ala cause loss of activity) are replaced by conservative substitution while non-essential amino acids can be deleted or replaced by any type of substitution. Guidelines for the determination of the deletions, replacements and substitutions are given in the Detailed Description Part of the specification.

The term “region” refers to a sequence in a specific location is the Lyn-kinase that corresponds to the positions selected from: 434 to 458 (termed: HJ loop); positions 318-336 (termed: αD region); position 305-313 (termed: B4-B5 region) and position 291-308 (termed: A-region).

The term “corresponding D-amino acid” refers to the replacement of the naturally occurring L-configuration of the natural amino acid residue by the D-configuration of the same residue.

The term “at least one peptidic backbone has been altered to a non-naturally occurring peptidic backbone” means that the bond between the N- of one amino acid residue to the C- of the next has been altered to non-naturally occurring bonds by reduction (to —CH₂—NH—), alkylation (methylation) on the nitrogen atom, or the bonds have been replaced by amidic bond, urea bonds, or sulfonamide bond, etheric bond (—CH₂—O—), thioetheric bond (—CH₂—S—), or to —CS—NH—,; The side chain of the residue may be shifted to the backbone nitrogen to obtain N-alkylated-Gly (a peptidoid).

The term “in reverse order” refers to the fact that the sequence of (a) to (f) may have the order of the amino acids as it appears in the native Lyn kinase from N- to the -C direction, or may have the reversed order (as read in the C- to N-direction) for example, if a subsequence of the HJ-loop of Lyn is GIVTYGK a sequence in a reverse order is KGYTVIG. It has been found that many times sequences having such a reverse order can have the same properties, in small peptides, as the “correct” order, probably due to the fact that the side chains, and not the peptidic backbones are those responsible for interaction with other cellular components. Particularly preferred, are what is termed “retro inverso” peptides—i.e. peptides that have both a reverse order as explained above, and in addition each and every single one of the amino acids, has been replaced by the non-naturally occurring D- amino acid counterpart, so that the net end result, as regards the positioning of the side chains, (the combination of reverse order and the change from L to D) is zero change. Such retro-inverso peptides, while having similar binding properties to the native peptide, were found to be resistant to degradation.

The present invention further concerns a method for the treatment of prostate cancer in a subject comprising administering to the subject a therapeutically effective amount of a compound comprising a sequence selected from:

(a) a sequence which is a continuous stretch of at least five amino acids present in Lyn-kinase in positions 434-458 (HJ loop);

(b) a sequence which is a continuous stretch of at least five amino acids present in Lyn kinase in positions 318-336 (αD region);

(c) a sequence which is a continuous stretch of at least five amino acids present in Lyn-kinase in positions 305-316 (B4-B5 region);

(d) a sequence which is a continuous stretch of at least five amino acids present in a native Lyn kinase in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant, and provided that the variant maintains the biological activity of the parent sequence of (a) to (d);

(f) a sequence of any one of (a) to (e) wherein at least one of the amino acids is replaced by the corresponding D- amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h).

The present invention also concerns use of a compound comprising a sequence selected from:

(a) a sequence which is a continuous stretch of at least five amino acids present in Lyn-kinase in positions 434-458 (HJ loop);

(b) a sequence which is a continuous stretch of at least five amino acids present in Lyn kinase in positions 318-336 (αD region);

(c) a sequence which is a continuous stretch of at least five amino acids present in Lyn-kinase in positions 305-316 (B4-B5 region);

(d) a sequence which is a continuous stretch of at least five amino acids present in a native Lyn kinase in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant and provided that the variant maintains the biological activity of the parent sequence of (a) to (d);

(f) a sequence of any one of (a) to (e) wherein at least one of the amino acids is replaced by the corresponding D- amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h); for the preparation of a medicament for the treatment of prostate cancer. The term “treatment of prostate cancer” includes at least one of the following: decrease in the rate of growth of the cancer (i.e. the cancer still grows but at a slower rate); cease of prostate cancer growth, i.e., stasis of the prostate cancer tumor occurs, and, in preferred cases, the prostate cancer tumor diminishes or is reduced in size. The term also concerns reduction in the number of metastasis, reduction in the number of new metastasis formed, slowing of the progression of the cancer from one stage to the other and decrease in angiogenesis induced by the cancer. In most preferred cases, the prostate cancer tumor is totally eliminated. This term also concern prevention for prophylactic situations or for those individuals who are susceptible to contracting prostate tumor cancer, the administration of said compounds will reduce the likelihood of the individual contrasting the disease. In preferred situations, the individual to whom the compound is administered does not contract the disease.

The term “prostate cancer” in the context of the present invention concerns both hormone responsive, as well as hormone refractory prostate cancer, as well as benign prostate hypertrophic conditions.

The present invention also concerns a method for obtaining of the most favorable compounds comprising the above sequences (a) to (i), for the reduction in the growth of prostate cancer cells.

Thus the present invention concerns a method for obtaining compounds for the treatment of prostate cancer, the method comprising:

(a) identifying peptide regions in Lyn-kinase that are in positions selected from: 434-458 (HJ-loop), 291-308 (A-region), 305-313 9B4-B5 region), 318-336 (αD region);

(b) synthesizing a plurality of compounds comprising a sequence selected from:

(b 1) a sequence corresponding to at least five continuous amino acid sequences of the HJ-loop, A-region, B4-B5 or αD region;

(b2) a variant of the sequence according to (b1) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant, and provided that the variant maintains the biological activity of the parent sequence of (a) to (d);

(b3) a sequence of (b1) or (b2) wherein one or more of the amino acids has been replaced by the corresponding D-amino acid;

(b4) a sequence of (b1), (b2) or (b3) wherein at least one of the peptidic backbone has been altered to a non-naturally occurring amino acid;

(b5) a sequence being the sequence of any one of (b1), (b2), (b3) or (b4) in a reverse order; and

(b6) a combination of two or more sequences of(b1)-(b5);

(c) testing the modulation activity of the compounds of (b) in a test assay for determining their activity in the reduction of growth of prostate cancer cells;

(d) selecting from the compounds of (c) those compounds which caused reduction of the growth of prostate cancer cells in the test assay as compared to the reduction in the same test assay in the absence of the compound; and

(e) producing the compounds of (d) thereby obtaining compounds for the reduction of prostate cancer cell growth.

Preferably, the amino acid sequence of (a) above should be in positions 434 to 458 of the Lyn-kinase, more preferably in position 436 to 441 of the Lyn-kinase.

The amount of compounds of the invention administered to the individual will depend on the type and severity of the disease (for example, hormone refractory vs. hormone responsive) and on the characteristics of the individual, such as general health, age, body weight and tolerance to drugs as well as on the mode of administration. The skilled artisan will be able to determine appropriate dosages depending on these and other factors. Typically, a therapeutically effective amount of the compound can range from about 1 mg per day to about 1000 mg per day for an adult. Preferably, the dosage ranges from about 1 mg per day to about 100 mg per day.

By a second aspect the present invention concerns a method for reduction of growth of cancer cells comprising administering to the cancer cells an effective amount of a LAST-inhibitor. The invention concerns methods for the treatment of prostate cancer comprising administering to a subject in need of such treatment a therapeutically effective amount of an inhibitor of LAST.

Any inhibitor of LAST can be administered to the individuals in the course of treating prostate cancer. Among the Lyn-tyrosine kinase inhibitors that can be employed are compounds comprising sequences derived from Lyn-kinase regions responsible for interaction with cellular components or variants of such sequences as described above, antibodies immunoreactive with Lyn-kinases, anti-sense nucleic acids that block expression of Lyn-kinases; negative-dominant Lyn tyrosine kinase genes which express Lyn-kinase proteins with reduced or non-existent biological activity, ribozymes that specifically cleave Lyn RNA and small organic molecules. Any of these inhibitors of Lyn kinase will inhibit the growth of prostate cancer in individuals.

Preferably the LAST inhibitors are compounds comprising sequences derived from regions of the Lyn-kinase which are responsible for interaction with other cellular components, especially with the substrate. As indicated above, it is assumed that peptides mimicking said regions, bind to the cellular components (such as substrates of the Lyn-kinase), and by this interrupt the interaction of the Lyn-kinase and the substrate, leading to inhibition of LAST.

More specifically, the LAST inhibitor is a compound comprising a sequence selected from:

(a) a sequence which is a continuous stretch of at least five amino acids present in Lyn-kinase in positions 434-458 (HJ loop);

(b) a sequence which is a continuous stretch of at least five amino acids present in Lyn kinase in positions 318-336 (αD region);

(c) a sequence which is a continuous stretch of at least five amino acids present in Lyn-kinase in positions 305-316 (B4-B5 region);

(d) a sequence which is a continuous stretch of at least five amino acids present in Lyn kinase in positions 291-308 (A-region);

(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified and/or up to 20% of the amino acids have been deleted, provided that at least 50% of the amino acids in the parent sequence of (a) to (d) are maintained unaltered in the variant, and provided that the variant maintains the biological activity of the parent sequence of (a) to (d);

(f) a sequence of any one of (a) to (e) wherein at least one of the amino acids is replaced by the corresponding D- amino acid;

(g) a sequence of any one of (a) to (f) wherein at least one of the peptidic backbones has been altered to a non-naturally occurring peptidic backbone;

(h) a sequence being the sequence of any one of (a) to (g) in reverse order; and

(i) a combination of two or more of the sequences of (a) to (h)

Specific examples are compounds which comprise any one of the sequences as specified in SEQ ID NO: 1-SEQ ID NO: 81, and which will be referred to in the following description with the following annotations: K055H007 (SEQ ID NO: 1); K055H101 (SEQ ID NO: 2); K055H104 (SEQ ID NO: 3); K055H108 (SEQ ID NO: 4); K055H10 (SEQ ID NO: 5); K055H104 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74), as specified in FIG. 2, or K055H719. SEQ ID NO: 75 as well as compounds comprising any of the following sequences: SEQ ID NO: 76 (HJ-full sequence); SEQ ID NO: 77 (HJ-subsequence); SEQ ID NO: 78 (αD-region-full sequence); SEQ ID NO: 79 (αD-subsequence); SEQ ID NO: 80 (B4-B5-full sequence) or SEQ ID NO: 81 (A-region-full sequence).

This invention also relates to the reduction of the growth of prostate cancer cells by administering one or more inhibitors of LAST to the prostate cancer cells. The administration of inhibitors of LAST to prostate cancer cells causes a reduction in the growth of these cells and, at least eventually, causes a reduction in the number of these cells. Again, any inhibitor of LAST will inhibit the growth of prostate cancer cells when delivered to these cells. The inhibitors include the compounds comprising the peptides from the regions defined above and their variants, antibodies, anti-sense nucleic acids, negative dominant LAST genes, and small organic molecules.

The term “Lyn-associated signal transduction (LAST)” refers to the level of signaling mediated by Lyn-kinase, which is best determined by determination of the phosphorylation level of at least one substrate in the lyn-signaling pathway which may be a direct substrate of Lyn (Lyn itself, CD19, CD79, Vav, Syk, Shc, PI3-kinase (p85), N-Myristoyltransferase (NMT), FAK, Protein Band 3,Syk,SLP-65, Tec protein tyrosine kinase,HSI) ) or a substrate of another kinase more downstream in the Lyn-kinase signaling pathway, such as MAP kinase, ERK, PKB.

The sequences which correspond to regions of Lyn, , in addition to their ability to reduce prostate cancer growth in individuals or their ability to inhibit the growth of prostate cancer cells, also are useful for generating antibodies that reduce prostate cancer growth and inhibit the growth of prostate cancer cells. The sequences act as antigenic agents for producing such antibodies. These antibodies, in turn, act as inhibitors of LAST, thereby reducing prostate cancer growth and inhibiting prostate cancer cell growth when they are administered to the individual with prostate cancer or to the prostate cancer cells.

### BRIEF DESCRIPTION OF THE DRAWINGS

FIG. 1 is a Table illustrating the amino acid sequences of the lyn-derived peptides.

FIGS. 2A-2B is a Table illustrating the sequences of the peptides K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74).

FIG. 3 is a graph showing the percent inhibition of proliferation of DU145 prostate cancer cells by increasing concentrations of five compounds of the invention K055H101 (SEQ ID NO: 2); K055H123 (SEQ ID NO: 18); K055H137 (SEQ ID NO: 26); K055H302 (SEQ ID NO: 61); K055H719 (SEQ ID NO: 75).

FIG. 4 is a graph showing the percent inhibition of proliferation of PC3 prostate cancer cells by increasing concentrations of five compounds of the invention: K055H101 (SEQ ID NO: 2); K055H123 (SEQ ID NO: 18); K055H137 (SEQ ID NO: 26); K055H302 (SEQ ID NO: 61); K055H719 (SEQ ID NO: 75).

FIG. 5 is a graphical representation of the change of prostate cancer tumor over a period of time for control animals and a group of animals to whom the peptide K055H302 (Bblac) (SEQ ID NO: 61) and K055H719 (Bblac) (SEQ ID NO: 75) had been administered. FIG. 5A—change defined as percentage of change from the initial volume, FIG. 5B—change defined as absolute change in size of tumor.

FIG. 6 Western blots showing the phosphorylation levels of several Lyn substrates in the presence and absence of compound of the invention K055H302 (SEQ ID NO: 61);

FIG. 7 Shows soft agar results of the effect of the compound of the invention K055H302 (SEQ ID NO: 61), on the invasiveness of DU-145 cells;

FIG. 8 shows co-immunoperciptation results of Lyn and its substrate Syk in the presence and absence of the compound of the invention K055H302 (SEQ ID NO: 61).

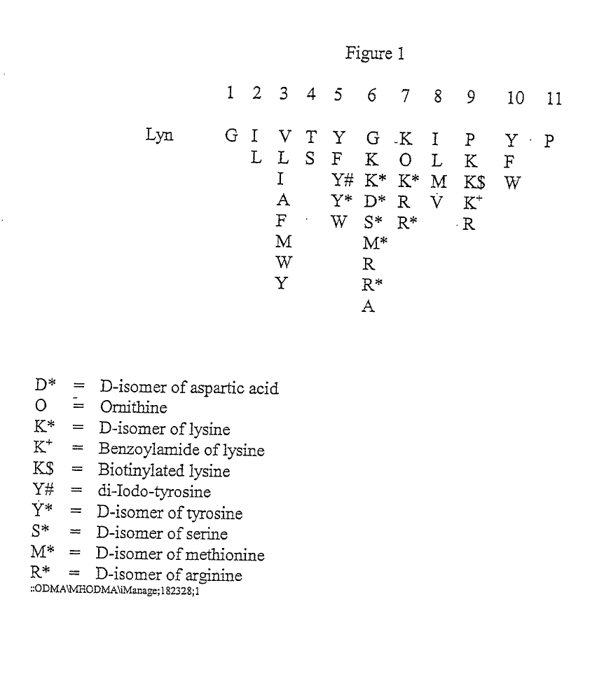

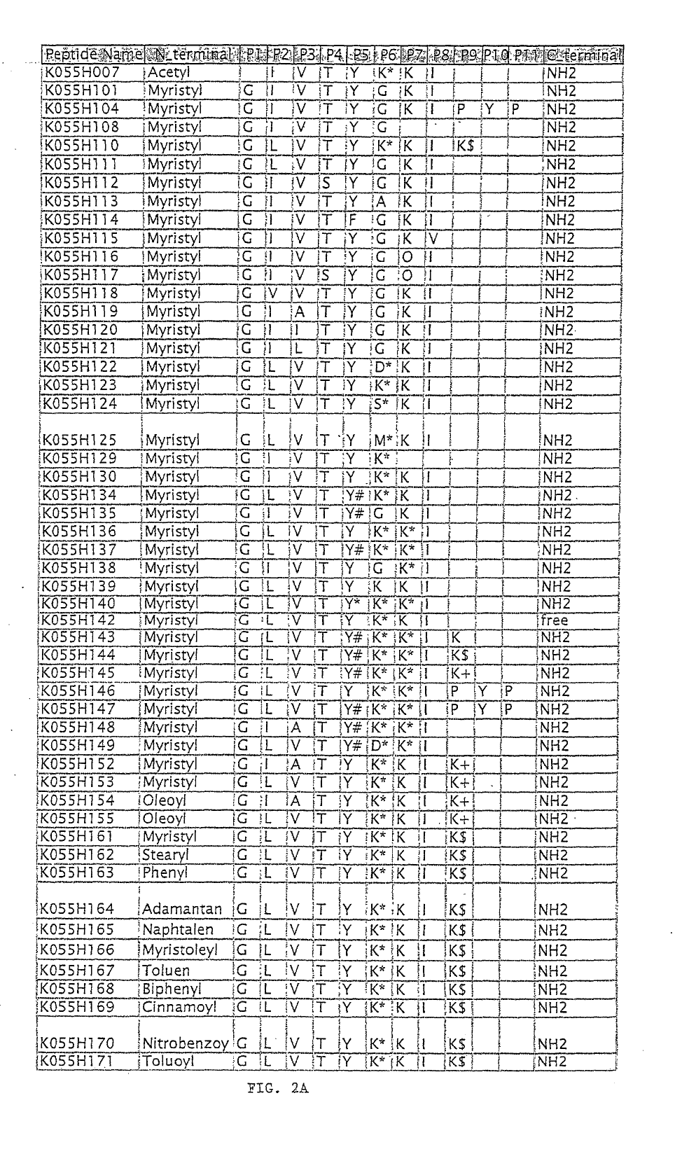

### DETAILED DESCRIPTION OF THE INVENTION

The present invention is based on the finding that Lyn-tyrosine kinase is active in prostate cells and that the reduction of the signal transduction associated with the kinase (as determined for example by the reduction of the phosphorylation of the kinase substrate) inhibits the growth of prostate cancer cells. Thus the administration of inhibitors of LAST, causes the inhibition of the signal transduction leading to the reduction in prostate cancer growth.

This invention is also directed to methods for inhibiting the growth of prostate cancer cells, whether within the body of an individual, in the prostate or in metastasis, or anywhere outside an individual's body, such as in an in vitro setting. These methods are directed to administering one or more inhibitors of LAST to the prostate cancer cells. The inhibitor or inhibitors is (are) administered in amounts that are effective in reducing the growth of the prostate cancer cells. When the inhibitors are administered to the prostate cancer cells, the cells stop proliferating (growing or dividing) as rapidly as they did in the absence of the inhibitors. In many instances, growth of the prostate cancer cells entirely ceases for example since the prostate cancer cells lose their viability and die. The growth retardation or death of the prostate cancer cells occurs because Lyn tyrosine kinase associated signal transduction is involved with growth and viability of prostate cancer cells.

Any inhibitor of LAST will thus serve to decrease the level of Lyn-associated signal transduction and thus will act to decrease growth of cancer cells. as will be explained below.

Small Molecule Inhibitors

Low molecular weight organic molecules can act as inhibitors of Lyn tyrosine kinase directly (by binding) and by this inhibit the LAST. Such low molecular weight organic molecules are known in the art. Exemplary of such compounds is the pyrazolo pyrimidine tyrosine kinase inhibitor PP1, or PP2 (see Schindler et al. “Crystal Structure of Hck in Complex with a Src Family-Selective Tyrosine Kinase Inhibitor”, Molecular Cell, Vol. 3, 639-648, May 1999, the pertinent contents of which are incorporated herein by reference). Other organic compound inhibitors of the Src family tyrosine kinases are known. Preferred low molecular weight organic molecules for use with the present invention are those that specifically inhibit the activity of Lyn tyrosine kinase.

Ribozymes That Specifically Cleave Lyn-RNA

A specific modulator of LAST is a ribozyme that is a catalytic oligonucleotide (typically RNA). The catalytic nucleotide can be tailored to specifically recognize, via hybridization, a specific mRNA region and thus cleave it and eliminate its expression. The ribozymes may be introduced to the cell as catalytic RNA molecules or as expression constructs for the expression of the catalytic RNA molecules.

Antisense LAST Inhibitors

Another type of inhibitor of LAST is anti-sense nucleic acids. The nucleic acids are single stranded ribonucleic or deoxyribonucleic acid strands which contain nucleotides joined together through normal sugar-phosphate bonds. Antisense sequences can inhibit production of Lyn protein by one of three mechanisms. By a first mechanism these antisense interfere with transcription as these antisense hybridize within the structural gene or in the regulatory gene thereof, that encodes for Lyn tyrosine kinase. This hybridization interrupts the transcription of Lyn tyrosine kinase gene into mRNA. Since proper transcription or expression is effectively blocked by the hybridization of the anti-sense nucleic acids to the DNA that contains the Lyn tyrosine kinase structural gene, the kinase production is decreased and as a result of the depletion of the kinase the LAST is inhibited.

A second mechanism is the binding of the antisense in the cytoplasm to the mRNA, thus interfering with the formation of a proper translation construct leading to inhibition of translation of the protein. This leads to the decrease in the amount of Lyn -kinase protein produced and thus to an inhibition of LAST.

A third mechanism is the formation of an mRNA-antisense duplex which leads to rapid degradation of mRNA duplex by RNases (such as Rnase H). All these mechanisms lead to production of smaller amounts of Lyn- produced by the prostate cancer cells than without the presence of these anti-sense nucleic acids, thus leading to LAST inhibition.

The particular nucleotides that are joined together to form the anti-sense sequence are those that are complementary to a region of the Lyn tyrosine kinase structural gene, or complementary to regulatory region of the gene sufficient to inhibit production of functional Lyn. These nucleotides of the anti-sense nucleic acids are specifically determined by the nucleotides of the target location and can easily be identified by the skilled practitioner once the sequence of the target location is established. The target location is a matter of choice to some extent. It lies within the region of the structural gene that encodes Lyn tyrosine kinase or in the regulatory coding region of the structure. The target location nucleotide sequence can easily be established by the skilled practitioner from publicly available information concerning the Lyn tyrosine kinase gene or can be obtained by routine examination of homologous genes coupled with standard molecular biology techniques.

By one option, the antisense is an oligonucleotide of several to several tens of nucleotides that are inserted into the cells. This is the preferred oligonucleotide in accordance with the invention. Typically the sequence is the first 20-25 nucleotides in the 5′ terminal of the Lyn cDNA (that are complementary to the mRNA). An example of such sequence is:

5′ atggga tgtataaaat caaaagggaa agac, or an RNA sequence as the above, wherein t has been replaced by u

Another option is the use of longer antisense sequences (up to several hundred nucleotides) by insertion into an expression vector, which can then transfected into the prostate cancer cell by various gene transfer technologies. If that case the full sequence of the Lyn can be used to construct a sequence which is complementary to it to produce a long antisense mRNA complementary to the native RNA. Finding the target of the kinase sequence to be used for antisense purposes may be carried out by screening through various overlapping sequences, or by use of various bioinformative software that can locate likely targets in a given gene and give several alternative sequences for producing antisense sequences that can eliminate production.

Negative Dominant Kinase Genes

Still another type of inhibitor of LAST is negative dominant Lyn tyrosine kinase genes. The presence of these genes in prostate cancer cells allows non-functional Lyn tyrosine kinase to be expressed to the exclusion of functional Lyn tyrosine kinase. The negative dominant Lyn tyrosine kinase in the prostate cancer cells is inhibitory of LAST activity because this kinase is non-functional. Non-functional kinases, by definition, have no kinase activity. Negative dominant Lyn tyrosine kinase genes are introduced into prostate cancer cells by gene transfer techniques, which are becoming increasingly more standard in the art (calcium precipitation, electrical discharge, physical injection, use of carriers such as recombinant vectors, etc.). The introduced negative dominant Lyn tyrosine kinase gene is incorporated in the prostate cancer cell genome. There, copies of it are passed to progeny cells. Since this Lyn-tyrosine kinase gene is negative dominant, it will be expressed in response to signals which induce Lyn tyrosine kinase expression rather than the active form of Lyn tyrosine kinase. Prostate cancer cells which have incorporated the negative dominant Lyn tyrosine kinase gene will not grow because the expressed Lyn tyrosine kinase is inactive. The negative dominant Lyn tyrosine kinase genes can be found in the art or can be produced by standard gene mutation techniques which are well known to skilled practitioners in the art. These genes can be suitably packaged for transgenic procedures by appropriate methods and materials known to the skilled practitioners.

A specific example of such a gene is a sequence wherein the codon lys 425 (AAA) in the region of the catalytic core encoding Lyn responsible to ATP-binding has been replaced with the Alanine or methione. Other examples are replacement of the codon of lys 275(AAA) by codon for Arg (CGU/C/A/G www.pnas.org/cgi/content/full/98/18/10172 or replacement of the codon for Tyr 397 (TAC) by codon for the Phe

(aaa/c http:/emboj.oupjournals.org/cgi/content/full/6/7/1610.

Antibodies Against Lyn for Inhibitor LAST:

A further type of inhibition of LAST is antibodies that are immunoreactive with Lyn tyrosine kinase. These antibodies bind to the kinase and thereby severely limit or prohibit its kinase activity or interrupt its interaction with other cellular components, all the above leading to LAST inhibition. The antibodies can be of any class or type. The binding site of the antibodies can be anywhere on the Lyn tyrosine kinase molecule provided the immunoreactive binding between the antibody and the kinase molecule results in a severe inhibition of LAST. The antibodies can be polyclonal or monoclonal and are produced by well-known techniques to the skilled practitioner by using the Lyn tyrosine kinase or immunogenic fragments thereof as the antigenic stimulus. The antibodies can be delivered to the prostate cancer cells by depositing suitable clonal cells, which produce the antibodies, into the individual who has prostate cancer or who is susceptible to contracting prostate cancer. These clonal cells secrete the antibodies into the bloodstream where they are carried to the prostate cancer cells for immunoreaction with the lien-tyrosine kinase molecules. Binding fragments of antibodies are also suitable provided they bind Lyn-tyrosine kinase with sufficient affinity that the activity of the kinase is at least severely limited. Alternatively, the antibodies or suitable binding fragments can be introduced into prostate cancer cells by any of a variety of techniques known to the skilled practitioner (physical injection, attachment to carriers that cross cell membranes, transgenic introduction into the prostate cancer cells for subsequent induction of expression, etc.). The secreted, introduced or expressed antibodies or suitable antibody fragments thereof immunoreactively bind to the Lyn-tyrosine kinase molecules, thereby inhibiting their activity. Commercially available anti-Lyn antibodies are available (Anti-Lyn (Santa Cruz, US) SC15 (44)).

Compounds Comprising Lyn Derived Peptides:

A further type of inhibitors of LAST is compounds comprising peptides, which herein are designated as “Lyn-derived peptides”. These compounds comprising or consisting of said Lyn-derived peptides are the preferred inhibitors of LAST, in accordance with the invention and thus are the preferred agents for the reduction of prostate cancer cell growth and for the treatment of of prostate cancer in an individual. The peptides apparently mimic a region in the kinase and thus bind to other cellular components with which the Lyn-kinase interacts (such as the kinase substrates). This binding interrupts the kinase-component interaction (especially kinase-substrate interaction) and thus inhibit LAST. It should be noted that Lyn-kinase may form dimmers with other Lyn-kinases, leading to trans-phosphorylation so that the substrate may be the Lyn-kinase itself

This LAST inhibition causes a reduction in the growth of prostate cancer tumors in vivo. Quite often the tumors are reduced in size and many times are eliminated altogether.

The peptides according to the above non-limiting theory mimic a region in the Lyn- tyrosine kinase that is involved in the interaction of the Lyn-tyrosine kinase with other cellular components that are part of the Lyn-associated signal transduction. Preferably, these cellular components are selected from: the substrates of Lyn-kinase, other kinases (which may be other Lyn-kinase proteases for trans-phosphorylation, or kinases of the same or different family), phosphatases, as well as co-factors and ATP. Thus, any peptide which mimics a part of the Lyn-kinase responsible for said interaction can bind to the cellular component, and thus inhibit the LAST.

The finding of such peptide involves routine screening of the Lyn-kinase regions as will be explained below.

Specific preferred regions of the Lyn kinase that the Lyn-derived peptides mimic are: the HJ-loop, αD-loop, A-region, and B4-B5 region, as defined above. It is clear that for interruption of the kinase-cellular component interaction there is no need to obtain a mimic of the full region of the kinase and a mimic of a subsequence may be sufficient to interrupt said interaction. It is further clear that the interruption may be caused by mimicking of any one of several smaller subsequences in the region and there is no necessity to mimic only one subsequence. It is further clear that for mimicking purposes it is not necessarily to obtain a sequence as present in the native kinase and variants of that sequence, that can faithfully copy the three dimensional structure of the region (when present in the full kinase), as well as copying the chemical characteristics of several of those side chains that bind to the substrate can also be used as mimics for interruption of the interaction. At times such variants may have better mimicking properties than the native sequence as the variation may help stabilize the mimic amino acid in a more favorable conformation.

The peptide derivative are short subsequences of at least five continuous amino acids obtained from the above sequences, as well as variants of the above sequences obtained by substitution of up to 40% of the amino acid with natural and non natural amino acids or with peptidomimetic moieties, and/or chemical modification of up to 40% of the amino acid residue, and/or deletions of up to 20% of the amino acids, provided that the peptide derivative has at least 50% of the amino acids as in the native peptide.

Most preferably, the sequence is at least five continuous amino acids obtained from the region of positions 434 to 458 HJ-loop, more preferably in positions 436 to 441 in said HJ-loop. The amino acid sequence may be a 5, 6, 7, 8, 9, 10, 11, 12, 13, 14, 15, 16 17 18 19 and 20 amino acids. The sequence may be the sequence of a naturally appearing in the HJ-loop. However, actual empirical experiments show that sequences having substitutions at times have better LAST inhibiting properties than native sequences. Therefore, in the scope of the present invention are also included variants of the native sequence of the at least five continuous amino acids from the HJ-loop, in which up to 40% of the amino acids has been substituted, and /or up to 40% have been chemically modified, and/or up to 20% have been deleted. In general, amino acids in the regions, and in particular the HJ-loop region, which are essential for LAST, should be either identical to those appearing in the native sequence, chemically modified or substituted by conservative substitutions (in the context of the present invention conservative substitutions also refer to substitutions by amino acids having the same steric properties, but when they replaced amino acid is charged, the substituted amino acid may be polar or hydrophobic as well).

The other positions in the sequence may be replaced by conservative, non-conservative substitutions both by naturally and non naturally occurring amino acids as well as by organic peptidomimetics.

Preferably, Gly in position should be replaced by a D-amino acid, most preferably D-lys, or D-Arg.

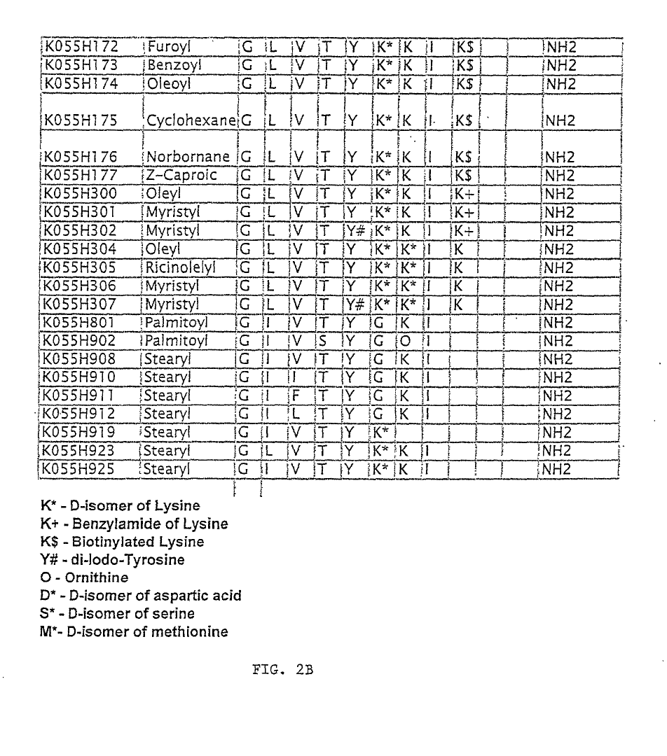

In this invention, particularly preferred compounds for inhibition of LAST are those labeled as K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H1 13 (SEQ ID NO: 8); K055H114 (SEQ ID NO: 9); K0551H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74), as specified in FIG. 2. Most preferable are K055H137 (SEQ ID NO: 26), K055302 (SEQ ID NO: 61) or K055H719, SEQ ID NO: 75, (or any of the SEQ ID NOS: 76-81 linked to a moiety for transfer across membranes).

Also included are peptides of SEQ ID NO: 1 to SEQ ID NO: 81, wherein 2-4 amino acids have been substituted, and/or 2-4 amino acids have been chemically substituted, preferably according to the guidelines given below.

Addition of Non-Peptidic Groups to One or to Both of the Terminals of the Sequences of (a) to (h) to Produce the Compound of the Invention Comprising a Lyn-Derived Peptide

Where the compound of the invention is a linear molecule, it is possible to place in any of its terminals various functional groups. The purpose of such a functional group may be for the improvement of the LAST inhibition. The functional groups may also serve for the purpose of improving physiological properties of the compound not related directly to LAST inhibition such as: improvement in stability, penetration (through cellular membranes or barriers), tissue localization, efficacy, decreased clearance, decreased toxicity, improved selectivity, improved resistance to repletion by cellular pumps, and the like. For convenience sake the free N-terminal of one of the sequences contained in the compounds of the invention will be termed as the N-terminal of the compound, and the free C-terminal of the sequence will be considered as the C-terminal of the compound (these terms being used for convenience sake). Either the C-terminus or the N-terminus of the sequences, or both, can be linked to a carboxylic acid functional groups or an amine functional group, respectively.

Suitable functional groups are described in Green and Wuts, “Protecting Groups in Organic Synthesis”, John Wiley and Sons, Chapters 5 and 7, 1991, the teachings of which are incorporated herein by reference. Preferred protecting groups are those that facilitate transport of the compound attached thereto into a cell, for example, by reducing the hydrophilicity and increasing the lipophilicity of the compounds these being an example for “a moiety for transport across cellular membranes ”.

These moieties can be cleaved in vivo, either by hydrolysis or enzymatically, inside the cell. (Ditter et al., J Pharm. Sci. 57:783 (1968); Ditter et al., J. Pharm. Sci. 57:828 (1968); Ditter et al., J. Pharm. Sci. 58:557 (1969); King et al., Biochemistry 26:2294 (1987); Lindberg et al., Drug Metabolism and Disposition 17:311 (1989); and Tunek et al., Biochem. Pharm. 37:3867 (1988), Anderson et al, Arch. Biochem. Biophys. 239:538 (1985) and Singhal et al., FASEB J 1:220 (1987)). Hydroxyl protecting groups include esters, carbonates and carbamate protecting groups. Amine protecting groups include alkoxy and aryloxy carbonyl groups, as described above for N-terminal protecting groups. Carboxylic acid protecting groups include aliphatic, benzylic and aryl esters, as described above for C-terminal protecting groups. In one embodiment, the carboxylic acid group in the side chain of one or more glutamic acid or aspartic acid residue in a compound of the present invention is protected, preferably with a methyl, ethyl, benzyl or substituted benzyl ester, more preferably as a benzyl ester.

In addition, a modified lysine residue can be added to the C-terminal of the compound to enhance biological activity. Examples of lysine modification include the addition of an aromatic substitute, such as benzoyl benzoic acid, dansyl-lysine various derivatives of benzoic acids (difluoro-, trifluromethy-, acetamido-, dimethyl-, dimethylamino-, methoxy-) or various derivatives of carboxylic acid (pyrazine-, thiophene-, pyridine-, indole-, naphthalene-, biphenyl,), or an aliphatic group, such as acyl, or a myristic or stearic acid, at the epsilon amino group of the lysine residue.

Examples of N-terminal protecting groups include acyl groups (—CO—R1) and alkoxy carbonyl or aryloxy carbonyl groups (-CO—O—R1), wherein R1 is an aliphatic, substituted aliphatic, benzyl, substituted benzyl, aromatic or a substituted aromatic group. Specific examples of acyl groups include acetyl, (ethyl)-CO—, n-propyl-CO—, iso-propyl-CO—, n-butyl-CO—, sec-butyl-CO—, t-butyl-CO—, hexyl, lauroyl, palmitoyl, myristoyl, stearyl, oleoyl phenyl-CO—, substituted phenyl-CO—, benzyl-CO— and (substituted benzyl)-CO—. Examples of alkoxy carbonyl and aryloxy carbonyl groups include CH3—O—CO—, (ethyl)-O—CO—, n-propyl-O—CO—, iso-propyl-O—CO—, n-butyl-O—CO—, sec-butyl-O—CO—, t-butyl-O—CO—, phenyl-O—CO—, substituted phenyl-O—CO— and benzyl-O—CO—, (substituted benzyl)- O—CO—. Adamantan, naphtalen, myristoleyl, tuluen, biphenyl, cinnamoyl, nitrobenzoy, toluoyl, furoyl, benzoyl, cyclohexane, norbornane, Z-caproic. In order to facilitate the N-acylation, one to four glycine residues can be present in the N-terminus of the molecule.

The carboxyl group at the C-terminus of the compound can be protected, for example, by an amide (i.e., the hydroxyl group at the C-terminus is replaced with —NH₂, —NHR₂ and —NR₂R₃) or ester (i.e. the hydroxyl group at the C-terminus is replaced with —OR₂). R₂ and R₃ are independently an aliphatic, substituted aliphatic, benzyl, substituted benzyl, aryl or a substituted aryl group. In addition, taken together with the nitrogen atom, R₂ and R₃ can form a C4 to C8 heterocyclic ring with from about 0-2 additional heteroatoms such as nitrogen, oxygen or sulfur. Examples of suitable heterocyclic rings include piperidinyl, pyrrolidinyl, morpholino, thiomorpholino or piperazinyl. Examples of C-terminal protecting groups include —NH₂, —NHCH₃, —N(CH₃)₂, —NH(ethyl), —N(ethyl)₂ —N(methyl) (ethyl), —NH(benzyl), —N(C1-C4 alkyl)(benzyl), —NH(phenyl), —N(C1-C4 alkyl) (phenyl), —OCH₃, —O—(ethyl), —O—(n-propyl), —O—(n-butyl), —O—(iso-propyl), —O—(sec- butyl), —O—(t-butyl), —O—benzyl and —O—phenyl.

Preferably the compounds includes in the N-terminal a hydrocarbon having a length of C₄-C₂₀ preferably C₆-C₁₈, most preferably C₈-C₁₆. Example of hydrophobic moieties are: aaystyl, stearyl, lauroyl, palmitoyl and acetyl etc. Other examoles are gernyl-gernyl,acetyl.

2. Finding a Shorter Subsequences of Lyn-Derived Peptides

As indicated, Lyn-derived peptides included in the compounds for inhibition of LAST, are obtained by finding which subsequence from the above regions (HJ-loop, A-region, αD-region, B4-B5 region) that inhibit LAST. Typically it is desired, for ease of synthesis and administration, to find the shortest sequence possible which is still active. In the following, the finding of the shortest sequence will be disclosed in connection with HJ-loop, but this description is applicable also to the other regions.

A shorter subsequence of the HJ-loop comprising a continuous stretch of at least five amino acid can be found by preparing a series of partially overlapping peptides each of 5-10 amino acids and each obtained by synthesizing a sequence that is one position removed from the previous sequence.

For example, the HJ-loop is in position 434 to 458, and it is to be desired to prepare 10 aa peptides, then the following, partially overlapping peptides are prepared, a peptide having the sequence 434-443, 435-444; 436-445 . . . 447-458. The LAST inhibiting activities of the subsequences is then determined in a test assay. The best 10-aa peptide is then chosen.

For checking whether the 10 aa peptide can be reduced in sequence, it is possible to either repeat the above procedure (preparing a series of partially overlapping peptides) using 5 aa long peptides that span the length of the chosen 10 aa peptide, or to shorten the 10 aa peptide by deleting alternatively from each terminal, an amino acid, and testing the LAST inhibiting activity of the progressively truncated peptides, until the optimal sequence of at least 5, at least 6, at least 7, at least 8, at least 9 aa peptide is obtained or until it is determined that longer sequences are required. As the HJ-loop (as well as the other regions) is relatively small, typically the number of different peptides to be tested is also small. For example, for an HJ-loop having a length of about 20 aa, there is a need to prepare only 12 peptides to find the optimal 8 aa peptide. After the best 8-aa peptide is obtained, it is possible to delete sequentially amino acids from one or both terminals of the 8 per peptide for obtaining the shortest sequence of 5, 6 or 7 aa that is still active. For these steps only 16 sequences have to be tested, so that by testing only 24 peptides it is possible to find such a shorter sequence.

3. Identifying Essential and Non-Essential Amino Acids in the Subsequence Chosen

A. Ala-Scan

Once the shorter continuous stretch of at least 5 (at least 6, 7, 8, 9, 10, 11 or 12) amino acids has been identified, as explained above, it is necessary to realize which of the amino acids in the stretch are essential (i.e. crucial for the kinase-associated signal transduction modulation) and which are non-essential. Without wishing to be bound by theory, in almost every native protein involved in interaction with other cellular components, some amino acids are involved with the interaction (essential amino acids) and some amino acids are not involved in the interaction (non-essential amino acids), for example since they are cryptic. A short peptide which is to mimic a region of the Lyn-kinase protein behaves in the same way as the region when present in the full kinase: some amino acids actually interact with the substrate (or other interacting components) and other amino acids merely serve to spatially position the interacting amino acids, but do not participate in the interaction with the other cellular components.

Essential amino acids have to be maintained (i.e. be identical to those appearing in the native kinase), or replaced by conservative substitutions (see definition below) to obtain variants of the peptides. Non-essential amino acids can be maintained, deleted, replaced by a spacer or replaced by conservative or non-conservative substitutions.

Identification of essential vs. non-essential amino acids in the peptide can be achieved by preparing several peptides that have a shorter sequence than the full region (see 2 above) in which each amino acid is sequentially replaced by the amino acid Ala (“Ala-Scan.”), or sequentially each amino acid is omitted (“omission-scan”). This allows to identify the amino acids which modulating activity is decreased by said replacement/omission (“essential”) and which are not decreased by said replacement/omission( “non-essential”) (Morrison et al., Chemical Biology 5:302-307, 2001). Another option for testing the importance of various peptides is by the use of site-directed mutagenesis. Other Structure-Activity-Relationship techniques may also be used.

B. 3D-analysis

Another strategy for finding essential vs. non-essential amino acids is by determining which aa of the A-region, in the 3D of the full kinase are exposed and which are cryptic. This can be done using standard software such as SPDB viewer ,“color by accessibility” of Glaxo- Welcome.

Typically cryptic aa are non-essential and exposed or partially exposed amino acids are more likely to be essential. However, if one wishes to “guess” theoretically which “non-conservative” substitutions in the cryptic region can be tolerated, a good guideline is to “check” on a 3D computer model of the fill kinase, whether a peptide superimposed on the full kinase and bearing those changes has still the overall structure of the region and more importantly, whether the exposed amino acids in the variants still overlap the positions of the exposed amino acids in the full kinase. Those non-conservative substitution, that when simulated on a computer 3D structure (for example using the Triphose™ software) do not cause drastic after action of the overall steps of the A-region (drastic shifting in the position of the exposed aa) are likely non-conservative replacements. Thus prior to experimental testing it is possible to reduce the number of tested candidates by computer simulation. Where the 3D structure of a specific kinase is not available in activating crystallography data, it is possible to obtain a “virtual” 3D structure of the kinase based on homology to known crystallographic structures using such progress such as CompSer™ (Tripose, USA).

4. Obtaining Variants

The sequence regions of the compound of the invention may be the native sequences obtained from the Lyn-kinase (preferably the shortest possible sequence from the region that has the highest activity), or alternatively variants of the native sequence obtained by deletion, (of non-essential amino acids) or substitution (only conservative substitutions in essential positions, both conservative and non-conservative of non-essential acids) or chemical modification.

4.1 Deletions and Insertions

Deletions can occur in particular of the “non-essential amino acids”. Additions may occur in particular at the N-terminal or the C-terminal of any of the amino acids of the sequence. No more than 20%, preferably 10% most preferably none of the amino acids should be deleted. Insertions should preferably be N-terminal or C-terminal to the sequence of (a) to (h) or between the several sequences linked to each other in (i). However other insertions or deletions are possible. Again, the feasibility of the deletions in creating a peptide which is a good mimic can be evaluated virtually by reverting to the 3D-module as described above, and finding which deletions still maintain the exposed side chains (when the peptide is superimposed on the kinase in the same positions.

4.2 Replacements

The variants can be obtained by replacement (termed also in the text as “substitution”) of any of the amino acids as present in the native kinase. As may be appreciated there are positions in the sequence that are more tolerant to substitutions than others, and in fact some substitutions may improve the activity of the native sequence. The determination of the positions may be realized using “Ala-Scan,” “omission scan” “site directed mutagenesis” or 3-D theoretical considerations as described in 3 above. Generally speaking the amino acids which were found to be “essential” should either be identical to the amino acids present in the native specific kinase or alternatively substituted by “conservative substitutions” (see bellow). The amino acids which were found to be “non-essential” might be identical to those in the native peptide, may be substituted by conservative or non-conservative substitutions, and may be deleted or replaced by a “spacers”.

The term “naturally occurring amino acid” refers to a moiety found within a peptide and is represented by —NH—CHR—CO—, wherein R is the side chain of a naturally occurring amino acid.

The term “non-naturally occurring amino acid” (amino acid analog) is either a peptidomimetic, or is a D or L residue having the following formula: —NH—CHR—CO—, wherein R is an aliphatic group, a substituted aliphatic group, a benzyl group, a substituted benzyl group, an aromatic group or a substituted aromatic group and wherein R does not correspond to the side chain of a naturally-occurring amino acid. This term also refers to the D-amino acid counterpart of naturally occurring amino acids. Amino acid analogs are well-known in the art; a large number of these analogs are commercially available. Many times the use of non-naturally occurring amino acids in the peptide has the advantage that the peptide is more resistant to degradation by enzymes which fail to recognize them.

The term “conservative substitution” in the context of the present invention refers to the replacement of an amino acid present in the native sequence in the specific kinase with a naturally or non-naturally occurring amino or a peptidomimetics having similar steric properties. Where the side-chain of the native amino acid to be replaced is either polar or hydrophobic, the conservative substitution should be with a naturally occurring amino acid, a non-naturally occurring amino acid or with a peptidomimetic moiety which is also polar or hydrophobic (in addition to having the same steric properties as the side-chain of the replaced amino acid). However where the native amino acid to be replaced is charged, the conservative substitution according to the definition of the invention may be with a naturally occurring amino acid, a non-naturally occurring amino acid or a peptidomimetic moiety which are charged, or with non-charged (polar, hydrophobic) amino acids that have the same steric properties as the side-chains of the replaced amino acids. The purpose of such a procedure of maintaining the steric properties but decreasing the charge is to decrease the total charge of the compound.

For example in accordance with the invention the following substitutions are considered as conservative: replacement of arginine by cytroline; arginine by glutamine; aspartate by asparagine; glutamate by glutamine.

As the naturally occurring amino acids are grouped according to their properties, conservative substitutions by naturally occurring amino acids can be easily determined bearing in mind the fact that in accordance with the invention replacement of charged amino acids by sterically similar non-charged amino acids are considered as conservative substitutions.

For producing conservative substitutions by non-naturally occurring amino acids it is also possible to use amino acid analogs (synthetic amino acids) well known in the art. A peptidomimetic of the naturally occurring amino acid is well documented in the literature known to the skilled practitioner.

When affecting conservative substitutions the substituting amino acid should have the same or a similar functional group in the side chain as the original amino acid.

The following are some non-limiting examples of groups of naturally occurring amino acids or of amino acid analogs are listed bellow. Replacement of one member in the group by another member of the group will be considered herein as conservative substitutions:

Group I includes leucine, isoleucine, valine, methionine, phenylalanine, serine, cysteine, threonine and modified amino acids having the following side chains: ethyl, n-butyl, —CH₂CH₂OH, —CH₂CH₂CH₂OH, —CH₂CHOHCH₃ and —CH₂SCH₃. Preferably Group I includes leucine, isoleucine, valine and methionine.

Group II includes glycine, alanine, valine, serine, cysteine, threonine and a modified amino acid having an ethyl side chain. Preferably Group II includes glycine and alanine.

Group III includes phenylalanine, phenylglycine, tyrosine, tryptophan, cyclohexylmethyl, and modified amino residues having substituted benzyl or phenyl side chains. Preferred substituents include one or more of the following: halogen, methyl, ethyl, nitro, methoxy, ethoxy and —CN. Preferably, Group III includes phenylalanine, tyrosine and tryptophan.

Group IV includes glutamic acid, aspartic acid, a substituted or unsubstituted aliphatic, aromatic or benzylic ester of glutamic or aspartic acid (e.g., methyl, ethyl, n-propyl iso-propyl, cyclohexyl, benzyl or substituted benzyl), glutamine, asparagine, CO—NH—alkylated glutamine or asparagine (e.g., methyl, ethyl, n-propyl and iso-propyl) and modified amino acids having the side chain —(CH₂)_{3—}COOH, an ester thereof (substituted or unsubstituted aliphatic, aromatic or benzylic ester), an amide thereof and a substituted or unsubstituted N-alkylated amide thereof. Preferably, Group IV includes glutamic acid, aspartic acid, glutamine, asparagine, methyl aspartate, ethyl aspartate, benzyl aspartate and methyl glutamate, ethyl glutamate and benzyl glutamate.

Group V includes histidine, lysine, arginine, N-nitroarginine, β-cycloarginine, μ-hydroxyarginine, N-amidinocitruline and 2-amino-4-15 guanidinobutanoic acid, homologs of lysine, homologs of arginine and ornithine. Preferably, Group V includes histidine, lysine, arginine, and omithine. A homolog of an amino acid includes from 1 to about 3 additional methylene units in the side chain.

Group VI includes serine, threonine, cysteine and modified amino acids having C1-C5 straight or branched alkyl side chains substituted with —OH or —SH. Preferably, Group VI includes serine, cysteine or threonine.

In this invention any cysteine in the original sequence or subsequence can be replaced by a homocysteine or other sulfhydryl-containing amino acid residue or analog. Such analogs include lysine or beta amino alanine, to which a cysteine residue is attached through the secondary amine yielding lysine-epsilon amino cysteine or alanine-beta amino cysteine, respectively.

The term “non-conservative substitutions” concerns replacement of the amino acid as present in the native Lyn-kinase by another naturally or non-naturally occurring amino acid, having different electrochemical and/or steric properties, for example as determined by the fact the replacing amino acid is not in the same group as the replaced amino acid of the native kinase sequence. Those non-conservative substitutions which fall under the scope of the present invention are those which still constitute a compound having kinase-associated signal transduction modulating activities. Because D-amino acids have hydrogen at a position identical to the glycine hydrogen side-chain, D-amino acids or their analogs can often be substituted for glycine residues, and are a preferred non-conservative substitution

A “non-conservative substitution” is a substitution in which the substituting amino acid (naturally occurring or modified) has significantly different size, configuration and/or electronic properties compared with the amino acid being substituted. Thus, the side chain of the substituting amino acid can be significantly larger (or smaller) than the side chain of the native amino acid being substituted and/or can have functional groups with significantly different electronic properties than the amino acid being substituted. Examples of non-conservative substitutions of this type include the substitution of phenylalanine or cycohexylmethyl glycine for alanine, isoleucine for glycine, or —NH—CH[(—CH₂)_{5—}COOH]—CO— for aspartic acid.

Alternatively, a functional group may be added to the side chain, deleted from the side chain or exchanged with another functional group. Examples of non-conservative substitutions of this type include adding an amine or hydroxyl, carboxylic acid to the aliphatic side chain of valine, leucine or isoleucine, exchanging the carboxylic acid in the side chain of aspartic acid or glutamic acid with an amine or deleting the amine group in the side chain of lysine or ornithine. In yet another alternative, the side chain of the substituting amino acid can have significantly different steric and electronic properties from the functional group of the amino acid being substituted. Examples of such modifications include tryptophan for glycine, lysine for aspartic acid and —(CH₂)₄COOH for the side chain of serine. These examples are not meant to be limiting.

As indicated above the non-conservative substitutions should be of the “non-essential” amino acids.

Preferably, the Lyn-kinase may be substituted by benzylamine groups, by biotinylation. Another substitution is di-iodinization of tyrosine. Liposomes may be substituted by D-isomers especially D-Lys residues. “Peptidomimetic organic moiety” can be substituted for amino acid residues in the compounds of this invention both as conservative and as non-conservative substitutions. These peptidomimetic organic moieties either replace amino acid residues of essential and non-essential amino acids or act as spacer groups within the peptides in lieu of deleted amino acids (of non-essential amino acids). The peptidomimetic organic moieties often have steric, electronic or configurational properties similar to the replaced amino acid and such peptidomimetics are used to replace amino acids in the essential positions, and are considered conservative substitutions. However such similarities are not necessarily required. The only restriction on the use of peptidomimetics is that the compounds retain their tissue-remodeling modulating activity as compared to compounds constituting sequence regions identical to those appearing in the native kinase.

Peptidomimetics are often used to inhibit degradation of the peptides by enzymatic or other degradative processes. The peptidomimetics can be produced by organic synthetic techniques. Examples of suitable peptidomimetics include D amino acids of the corresponding L amino acids, tetrazol (Zabrocki et al, J Am. Chem. Soc. 110:5875-5880 (1988)); isosteres of amide bonds (Jones et al., Tetrahedron Lett. 29: 3853-3856 (1988));

LL-3-amino-2-propenidone-6-carboxylic acid (LL-Acp) (Kemp et al., J Org. Chem. 50:5834-5838 (1985)). Similar analogs are shown in Kemp et al., Tetrahedron Lett. 29:5081-5082 (1988) as well as Kemp et al, Tetrahedron Lett. 29:5057-5060 (1988), Kemp et al., Tetrahedron Lett. 29:4935-4938 (1988) and Kemp et al., J Org. Chem. 54:109-115 (1987). Other suitable peptidomimetics are shown in Nagai and Sato, Tetrahedron Lett. 26:647-650 (1985); Di Maio et al., J Chem. Soc. Perkin Trans., 1687 (1985); Kahn et al., Tetrahedron Lett. 30:2317 (1989); Olson et al., J Am. Chem. Soc. 112:323-333 (1990); Garvey et al., J Org. Chem. 56:436 (1990). Further suitable peptidomimetics include hydroxy-1,2,3,4-tetrahydroisoquinoline- 3-carboxylate (Miyake et al., J Takeda Res. Labs 43:53-76 (1989)); 1,2,3,4-tetrahydro- isoquinoline-3-carboxylate (Kazmierski et al., J Am. Chem. Soc. 133:2275-2283 (1991)); histidine isoquinolone carboxylic acid (HIC) (Zechel et al., Int J Pep. Protein Res. 43 (1991)); (2S, 3S)-methyl-phenylalanine, (2S, 3R)-methyl-phenylalanine, (2R, 3S)-methyl-phenylalanine and (2R, 3R)-methyl-phenylalanine (Kazmierski and Hruby, Tetrahedron Lett. (1991)).

4.3 Chemical Modifications

In the present invention the side amino acid residues appearing in the native sequence may be chemically modified, i.e. changed by addition of functional groups. The modification may be in the process of Lyn-synthesis of the molecule, i.e. during elongation of the amino acid chain and amino acid, i.e. a chemically modified amino acid is added. However, chemical modification of an amino acid when it is present in the molecule or sequence (“in situ” modification) is also possible.

The amino acid of any of the sequence regions of the molecule can be modified (in the peptide conceptionally viewed as “chemically modified”) by carboxymethylation, acylation, phosphorylation, glycosylation or fatty acylation. Ether bonds can be used to join the serine or threonine hydroxyl to the hydroxyl of a sugar. Amide bonds can be used to join the glutamate or aspartate carboxyl groups to an amino group on a sugar (Garg and Jeanloz, Advances in Carbohydrate Chemistry and Biochemistry, Vol. 43, Academic Press (1985); Kunz, Ang. Chem. Int. Ed. English 26:294-308 (1987)). Acetal and ketal bonds can also be formed between amino acids and carbohydrates. Fatty acid acyl derivatives can be made, for example, by free amino group (e.g., lysine) acylation (Toth et al., Peptides: Chemistry, Structure and Biology, Rivier and Marshal, eds., ESCOM Publ., Leiden, 1078-1079 (1990)).

4.4 Cyclization of the Molecule

The present invention also includes cyclic compounds which are cyclic molecules.

A “cyclic molecule ” refers, in one instance, to a compound of the invention in which a ring is formed by the formation of a peptide bond between the nitrogen atom at the N-terminus and the carbonyl carbon at the C-terminus. “Cyclized” also refers to the forming of a ring by a covalent bond between the nitrogen at the N-terminus of the compound and the side chain of a suitable amino acid in the sequence present therein, preferably the side chain of the C-terminal amino acid. For example, an amide can be formed between the nitrogen atom at the N-terminus and the carbonyl carbon in the side chain of an aspartic acid or a glutamic acid. Alternatively, the compound can be cyclized by forming a covalent bond between the carbonyl at the C-terminus of the compound and the side chain of a suitable amino acid in the sequence contained therein, preferably the side chain of the N-terminal amino acid. For example, an amide can be formed between the carbonyl carbon at the C-terminus and the amino nitrogen atom in the side chain of a lysine or an ornithine. Additionally, the compound can be cyclized by forming an ester between the carbonyl carbon at the C-terminus and the hydroxyl oxygen atom in the side chain of a serine or a threonine.

“Cyclized” also refers to forming a ring by a covalent bond between the side chains of two suitable amino acids in the sequence present in the compound, preferably the side chains of the two terminal amino acids. For example, a disulfide can be formed between the sulfur atoms in the side chains of two cysteines. Alternatively, an ester can be formed between the carbonyl carbon in the side chain of, for example, a glutamic acid or an aspartic acid, and the oxygen atom in the side chain of, for example, a serine or a threonine. An amide can be formed between the carbonyl carbon in the side chain of, for example, a glutamic acid or an aspartic acid, and the amino nitrogen in the side chain of, for example, a lysine or an omithine.

In addition, a compound can be cyclized with a linking group between the two termini, between one terminus and the side chain of an amino acid in the compound, or between the side chains to two amino acids in the peptide or peptide derivative. Suitable linking groups are disclosed in Lobl et al., WO 92/00995 and Chiang et al., WO 94/15958, the teachings of which are incorporated into this application by reference.

Methods of cyclizing compounds having peptide sequences are described, for example, in Lobl et al., WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or omithine, for example) or acetamidomethyl (Acm) (for the sulfhydryl of cysteine) protecting groups. OAI and Aloc are easily removed by Pd and Acm is easily removed by iodine treatment.

5. Pharmaceutical Compositions and Therapeutical Methods of Treatment

The inhibitor of LAST of the present invention or the compounds for reduction of growth of prostate cancer cells can be used as active ingredients (together with a pharmaceutically acceptable carrier) to produce a pharmaceutical composition. The pharmaceutical composition may comprise one, or a mixture of two or more of the different LAST inhibitors of the invention in an acceptable carrier.

The pharmaceutical composition should be used for the treatment of a prostate cancer (hormone responsive or hormone refractory) as well as for the treatment of benign prostate hypertrophy.

The LAST inhibitors of the present invention can be administered parenterally. Parenteral administration can include, for example, systemic administration, such as by intramuscular, intravenous, subcutaneous, or intraperitoneal injection. Compounds which resist proteolysis can be administered orally, for example, in capsules, suspensions or tablets. The compound can also be administered by inhalation or insufflations or via a nasal spray.

The LAST inhibitors can be administered to the individual in conjunction with an acceptable pharmaceutical carrier as part of a pharmaceutical composition for treating the diseases discussed above. Suitable pharmaceutical carriers may contain inert ingredients which do not interact with the compounds. Standard pharmaceutical formulation techniques may be employed such as those described in Remington's Pharmaceutical Sciences, Mack Publishing Company, Easton, Pa. Suitable pharmaceutical carriers for parenteral administration include, for example, sterile water, physiological saline, bacteriostatic saline (saline containing about 0.9% mg/ml benzyl alcohol), phosphate-buffered saline, Hank's solution, Ringer's-lactate and the like. Methods for encapsulating compositions (such as in a coating of hard gelatin or cyclodextran) are known in the art (Baker, et al, Controlled Release of Biological Active Agents, John Wiley and Sons, 1986). The formation may be also resources for administration to bone, or in the form of salve, solution, ointment, etc. for topical administration.

The pharmaceutical compositions may also be administered in conjunction with other modes of therapy (chemotherapy, radiotherapy) routinely used in the treatment of prostate cancer.

A “therapeutically effective amount” is the quantity of compound which results in an improved clinical outcome as a result of the treatment compared with a typical clinical outcome in the absence of the treatment. An “improved clinical outcome” results in the individual with the disease experiencing fewer symptoms or complications of the disease, including a longer life expectancy, as a result of the treatment. With respect to cancer, an “improved clinical outcome” includes a longer life expectancy. It can also include slowing or arresting the rate of growth of a tumor, causing a shrinkage in the size of the tumor, a decreased rate of metastasis and/or improved quality of life (e.g., a decrease in physical discomfort or an increase in mobility).

6. Determination of LAST Inhibiting Activity

It should be appreciated that some of the compounds that comprise sequences (a)-(i) above are better LAST inhibitors than others and/or some of the compounds are better than others in reduction of prostate cancer cell growth. Some of the conservative substitutions in the essential positions may diminish the inhibiting, while other such conservative substitution in the essential positions may improve these inhibiting activities. The same is true also for deletions, substitutions (both conservative and non-conservative) in non-essential positions, as well as to chemical modifications (in any position) or insertions. In addition the type and size of the non-amino acid portion of the compounds, such as a hydrophobic moiety in one of its terminals may diminish or increase the LAST inhibiting activities. The LAST inhibiting activities that can be determined for example by using one of the assays stipulated below.

6.1 Cellular Assays

It can be readily determined whether a compound modulates the activity of a LAST by incubating the compound with cells which have one or more cellular activities controlled by the LAST. Examples of these cellular activities include cell proliferation, cell differentiation, cell morphology, cell survival or apoptosis, cell response to external stimuli, gene expression, lipid metabolism, glycogen or glucose metabolism and mitosis. The cells are incubated with the candidate compound to produce a test mixture under conditions suitable for assessing the level of the LAST. The activity of the LAST is assessed and compared with a suitable control, e.g., the activity of the same cells incubated under the same conditions in the absence of the candidate compound (or in the presence of a control compound). A lesser activity of LAST in the test mixture compared with the control indicates that the candidate compound inhibits LAST.

Suitable cells for the assay include normal cells which express the Lyn-kinase (such as B-cells), cells which have been genetically engineered to express a Lyn-kinase, malignant cells expressing a Lyn-kinase or immortalized cells that express the kinase.

Conditions suitable for assessing activity include conditions suitable for assessing a cellular activity or function under control of the LAST pathway. Generally, a cellular activity or function can be assessed when the cells are exposed to conditions suitable for cell growth, including a suitable temperature (for example, between about 30° C. to about 42° C.) and the presence of the suitable concentrations of nutrients in the medium (e.g., amino acids, vitamins, growth factors or of specific activators such as cytokines, hormones and the like).

For example, the proliferation of prostate cancer cells may be determined as in Example 2 below, i.e., determination of proliferation (for example as determined by methylene-blue dye assay) of prostate cancer cell lines such as DU-145 and PC3.

Another cellular assay is for determining the change of invasiveness of prostate cancer cells (such as DU-145 and PC-3) by using a soft agar assay, as specified in Example 4 below.

6.2 Phosphorylation of Substrates (in Cellular or Cell Free Assays)

It is possible to assess the LAST activity and the changes in this LAST as compared to control, by determining the phosphorylation level of the substrate proteins of the Lyn kinase. Examples of possible Lyn substrates are: (Lyn itself, CD19, CD79, Vav, Syk, Shc, P13-kinase (p85), N-Myristoyltransferase (NMT), FAK, Protein Band 3,Syk,SLP-65, Tec protein tyrosine kinase,HSI). Cells known to express the Lyn-kinase such as for example B-lymphocytes are incubated with a candidate compound for inhibiting the LAST and are activated. Then the cells are lysed, the protein content of the cells is obtained and separated on a gel. The substrates can be identified by use of suitable molecular weight markers, or by using suitable antibodies, reactive against Lyn, CD19, CD79, Syk, Vav, P13 kinase (p85), Shc, etc. The level of phosphorylation of the substrate may be determined by suing labeled anti-Tyr antibodies. Alternatively, the suitable substrate may be immuno-precipitated using antibodies. The level of substrate phosphorylation in the immuno- precipitate can be determined by using anti-phosphotyrosine antibodies (see Fujimoto et al., Immunity, 13:47-57 (2000)).

By another option, phosphorylation may be determined in a cell-free system by incubating a mixture comprising Lyn-kinases, the substrate of the kinase and candidate molecules for inhibiting LAST in the presence of ATP under conditions enabling, phosphorylation. The proteins are then subjected to gel separation, transferred to nitrocellulose where the substrate band is identified by antibody or molecular weight marker followed by immunoblotting by anti-phosphotyrosine antibody. Alternatively it is possible to use [γ-³² P] ATP and quantify the amount of radioactivity incorporated in the substrate (See Fujimoto et al, The J of Immunol. 7088-7094 (1999). Assays concerning phosphorylation of substances can be seen in Example 5.

6.3. Tissue or in vivo Assay

Suitable assays for determining inhibition of LAST can be by inducing prostate tumor in an experimental animal, by implanting prostate cell lines (for example Du-145, PC-3) in an experimental animal such as nude mice (subcutaneous) and then testing the effect of the candidate compound on one of the following: tumor size (decease in size, stasis or decreased growth rates as compared to control), progression of tumor to advanced stages (determined by histological techniques), survival of animals, spread of metastasis, angiogenesis and the like. Such an assay is shown in Example 3 below.

7. Preparation of Antibodies

The Lyn-derived peptides of the present invention can be useful in the preparation of specific antibodies against Lyn tyrosine kinase. Suitable antibodies can be raised against a Lyn peptide by conjugating the peptide to a suitable carrier, such as keyhole limpet hemocyanin or serum albumin; polyclonal and monoclonal antibody production can be performed using any suitable technique. A variety of methods have been described (see e.g., Kohler et al, Nature, 256:495-497 (1975) and Eur. J Immunol. 6:511-519 (1976); Milstein et al., Nature 266: 550-552 5 (1977); Koprowski et al., U.S. Pat. No. 4,172,124; Harlow, E. and D. Lane, 1988, Antibodies: A Laboratory Manual, (Cold Spring Harbor Laboratory: Cold Spring Harbor, N.Y.); Current Protocols In Molecular Biology, Vol. 2 (Supplement 27, Summer 1994), Ausubel, F. M. et al., Eds., (John Wiley & Sons: New York, N.Y.), Chapter 11, (1991)). Generally, a hybridoma can be produced by fusing a suitable immortal cell line (e.g., a myeloma cell line such as SP2/0) with antibody producing cells. The antibody producing cell, preferably those of the spleen or lymph nodes, can be obtained from animals immunized with the antigen of interest. The fused cells (hybridomas) can be isolated using selective culture conditions, and cloned by limiting dilution. Cells which produce antibodies with the desired specificity can be selected by a suitable assay (e.g., ELISA).

The antibodies can be used to determine if an intracellular lyn tyrosine kinase is present in the cytoplasm of the cell. A lysate of the cell is generated (for example, by treating the cells with sodium hydroxide (0.2 N) and sodium dodecyl sulfate (1%) or with a non-ionic detergent like NP-40, centrifugating and separating the supernatant from the pellet), and treated with anti-lyn peptide antibody specific for lyn tyrosine kinase. The lysate is then analyzed, for example, by Western blotting or immunoprecipitation for complexes between lyn tyrosine kinase and antibody. Anti-lyn peptide antibodies can be utilized for the study of the intracellular distribution (compartmentalization) of lyn tyrosine kinase under various physiological conditions via the application of conventional immunocytochemistry such as immunofluorescence, immunoperoxidase technique and immunoelectron microscopy, in conjunction with the specific anti-lyn peptide antibody.

Antibodies reactive with the lyn peptides are also useful to detect and/or quantitate the lyn tyrosine kinase in a sample, or to purify the lyn tyrosine kinase (e.g., by immunoaffinity purification).

The lyn-derived peptides of the present invention can also be used to identify ligands which interact with lyn tyrosine kinase and which inhibit the activity of lyn tyrosine kinase. For example, an affinity column can be prepared to which a lyn peptide is covalently attached, directly or via a linker. This column, in turn, can be utilized for the isolation and identification of specific ligands which bind the lyn peptide and which will also likely bind the lyn tyrosine kinase. The ligand can then be eluted from the column, characterized and tested for its ability to inhibit lyn tyrosine kinase function.

Peptide sequences in the compounds of the present invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Merrifield, J. Am. Chem. Soc. 88:2149 (1963); Meienhofer, Hormonal Proteins and Peptides, C.H. Li, Ed., Academic Press, 1983, pp. 48-267; and Barany and Merrifield, in The Peptides, E. Gross and J. Meienhofer, Eds., Academic Press, New York, 1980, pp. 3-285. Methods of solid phase peptide synthesis are described in Merrifield, R. B., Science, 232: 341 (1986); Carpino, L. A. and Han, G. Y., J Org. Chem., 37: 3404 (1972); and Gauspohl, H. et al, Synthesis, 5:315 (1992)). The teachings of these references are incorporated herein by reference.

Methods of cyclizing compounds having peptide sequences are described, for example, in Lobl et al, WO 92/00995, the teachings of which are incorporated herein by reference. Cyclized compounds can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or omithine, for example) or acetamidomethyl (Acm) (for the sulfhydryl of cysteine) protecting groups. OAI and Aloc are easily removed by Pd and Acm is easily removed by iodine treatment.

8. Preparation of the Compounds

Peptide sequences for producing any of the sequence of the compounds of the invention may be synthesized by solid phase peptide synthesis (e.g., t-BOC or F-MOC) method, by solution phase synthesis, or by other suitable techniques including combinations of the foregoing methods. The t-BOC and F-MOC methods, which are established and widely used, are described in Aarifield, J Am. Chem. Soc., 88:2149 (1963); Meienhofer, Hormonal Proteins and Peptides, C. H. Li, Ed., Academic Press, 1983, pp. 48-267; and Barany and Aarifield, in The Peptides, E. Gross and J. Meienhofer, Eds., Academic Press, New York, 1980, pp.3-285. Methods of solid phase peptide synthesis are described in Aarifield, R. B., Science, 232:341 (1986); Carpino, L. A. and Han, G. Y., J Org Chem., 37:3404 (1972); and Gauspohl, H. et al., Synthesis, 5:315 (1992)). The teachings of these references are incorporated herein by reference.

As indicated above the compounds of the invention may be prepared utilizing various peptidic cyclizing techniques. Methods of cyclizing compounds having peptide sequences are described, for example, in Lobl et al., WO 92/00995, the teachings of which are incorporated herein by reference. Cyclized molecules can be prepared by protecting the side chains of the two amino acids to be used in the ring closure with groups that can be selectively removed while all other side-chain protecting groups remain intact. Selective deprotection is best achieved by using orthogonal side-chain protecting groups such as allyl (OAI) (for the carboxyl group in the side chain of glutamic acid or aspartic acid, for example), allyloxy carbonyl (Aloc) (for the amino nitrogen in the side chain of lysine or ornithine, for example) or acetamidomethyl (Acm) (for the sulfhydryl of cysteine) protecting groups. OAI and Aloc are easily removed by Pd and Acm is easily removed by iodine treatment.

Other modes of cyclization (beyond N- to C- terminal cyclization) may include: N- to backbone cyclization, C- to backbone cyclization, N- to side chain cyclization, C- to side chain cyclization, backbone to side chain cyclization, backbone to backbone cyclization and side chain to side chain cyclization.

### EXAMPLE 1

### Preparation of Compounds Comprising Lyn-Derived Peptides

The compounds of this invention can be synthesized utilizing a 430A Peptide Synthesizer from Applied Biosystems using F-Moc technology according to manufacturer's protocols. Other suitable methodologies for preparing peptides are known to person skilled in the art. See e.g., Merrifield, R. B., Science, 232: 341 (1986); Carpino, L. A., Han, G. Y., J Org. Chem., 37: 3404 (1972); Gauspohl, H., et al., Synthesis, 5: 315 (1992)). The teachings of which are incorporated herein by reference.

Rink Amide Resin [4(2′,4′ Dimethoxyphenyl-FMOC amino methyl) phenoxy resin] was used for the synthesis of C-amidated peptides. The alpha-amino group of the amino acid was protected by an FMOC group, which was removed at the beginning of each cycle by a weak base, 20% piperidine in N-methylpyrrolidone (NMP). After deprotection, the resin was washed with NMP to remove the piperidine. In situ activation of the amino acid derivative was performed by the FASTMOC Chemistry using HBTU (2(1-benzo- triazolyl-1-yl)-1,1,3,3-tetramethyluronium) dissolved in HOBt (1-hydroxy- benzotriazole) and DMF (dimethylformamide). The amino acid was dissolved in this solution with additional NMP. DIEA (diisopropylethylamine) was added to initiate activation. Alternatively, the activation method of DCC (dicycbohexylcarbodiimide) and HOBL was utilized to form an HOBt active ester. Coupling was performed in NMP. Following acetylation of the N-terminus (optional), TFA (trifluoroacetic acid) cleavage procedure of the peptide from the resin and the side chain protecting groups was applied using 0.75 g crystalline phenol; 0.25 ml EDT (1,2-ethandithiol); 0.5 ml thioanisoie; 0.5 ml D.I. H₂O; 10 ml TFA.

### EXAMPLE 2

### Inhibition of Proliferation of Prostate Cancer Cells In Vitro by Incubation with Compounds Comprising Lyn-Derived Peptides

Human prostate cancer cell lines PC3 and DU145 were obtained from the American Type Culture Collection (ATCC No. 1435-CRL and 81-HTB). These cell lines were grown in RPMI 1640 medium supplemented with penicillin (100 U/ml), streptomycin (100 μg/ml), glutamine (2 mM) and 10% endotoxin free bovine cell serum (Hyclone).

A suspension of the cells at 2×10⁴ cells/ml was prepared in the above described culture medium and distributed 0.180 ml per well (about 4000 cells/well) in the wells of 96 well, flat bottom, tissue culture microtiter plates.

A series of compounds stock solutions were prepared by diluting a 10 mM solution of the compound in 100% DMSO with phosphate buffered saline (PBS) containing 0.1% bovine serum albumin (BSA) to a concentration of 400 μM. These solutions were labeled DMSO. In many instances, 40 μl of the 10 compound in DMSO solution was mixed with 160 μl of 2M NH₄HCO₃ and heated for 40 minutes at 100° C. The resultant solution was then diluted to 400 μM in PBS containing 0.1% BSA. These compounds stock solutions were labeled “tbi”. The concentration of compound in each stock solution was adjusted to nine times the desired concentration of the compound in the assay mixture. 0.020 ml of each compound stock solution was added to the corresponding wells about 2 hours after prostate cancer cell addition, with six replicates for each concentration. In addition, PBS containing 0.1% BSA solution with no added compound was used as a control. The plates were incubated for 72-80 hours at 37° C. in a 10% CO₂ humidified incubator. This formulation was termed “tbi”, and served as a vehicle and as control.

The plates were labeled and the medium discarded. The wells were fixed with 4% formaldehyde PBS (PBS buffered with 10% formalin from Fisher Scientific; Catalog No. HC200-1) ( 0.2 ml/well) for at least 30 minutes. The wells were washed one time with borate buffer (0.2 ml/well) (0.1M, pH 8.5). Freshly filtered 1% methylene blue solution (0.60 ml/well) was then added to the wells and incubated for 10 minutes at room temperature. The wells were then washed five times with tap water, after which the wells were dried completely. 0.20 ml/well of 0.1 N HCl was added to extract the color. After overnight extraction, the O.D. was read at 630 nm to determine the number of cells per well. The procedure for counting cells is described in greater detail in Oliver et al. J Cell Sci., 92: 513 (1989), the teachings of which are incorporated herein by reference.

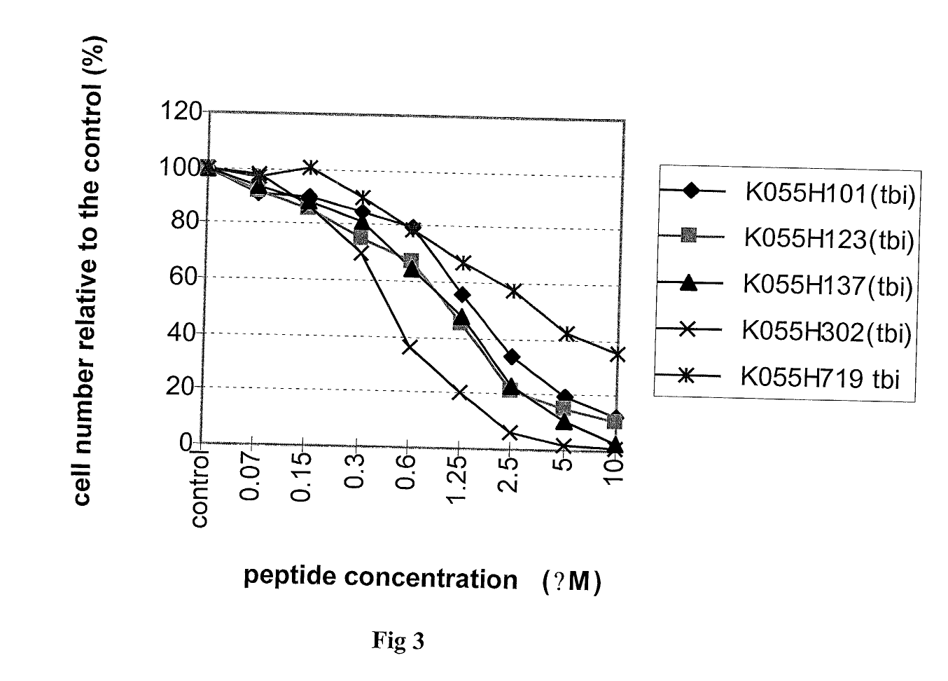

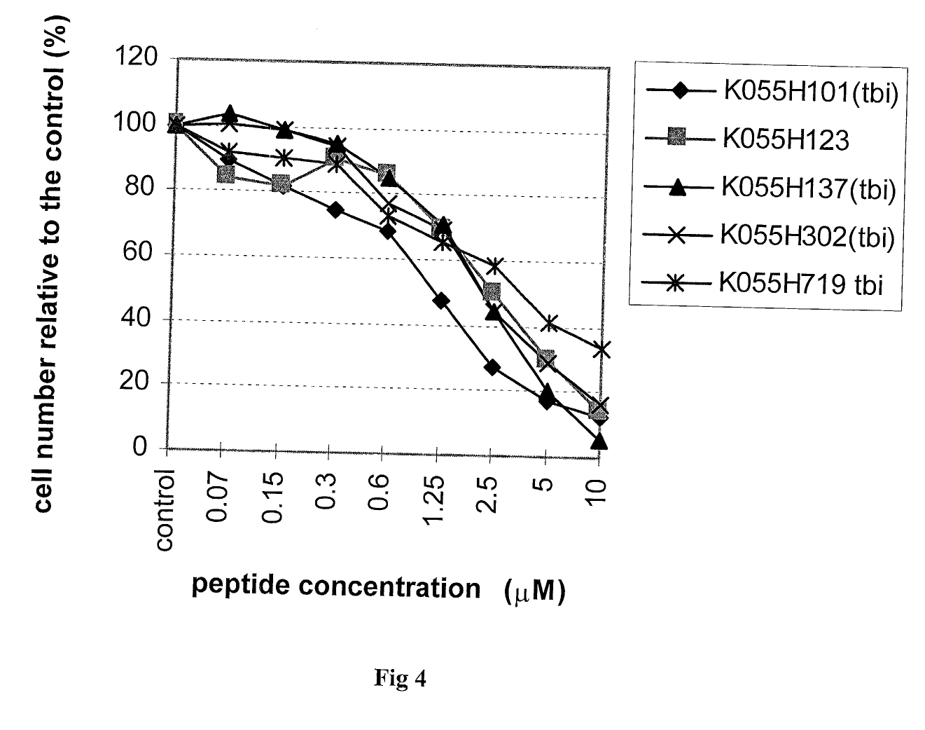

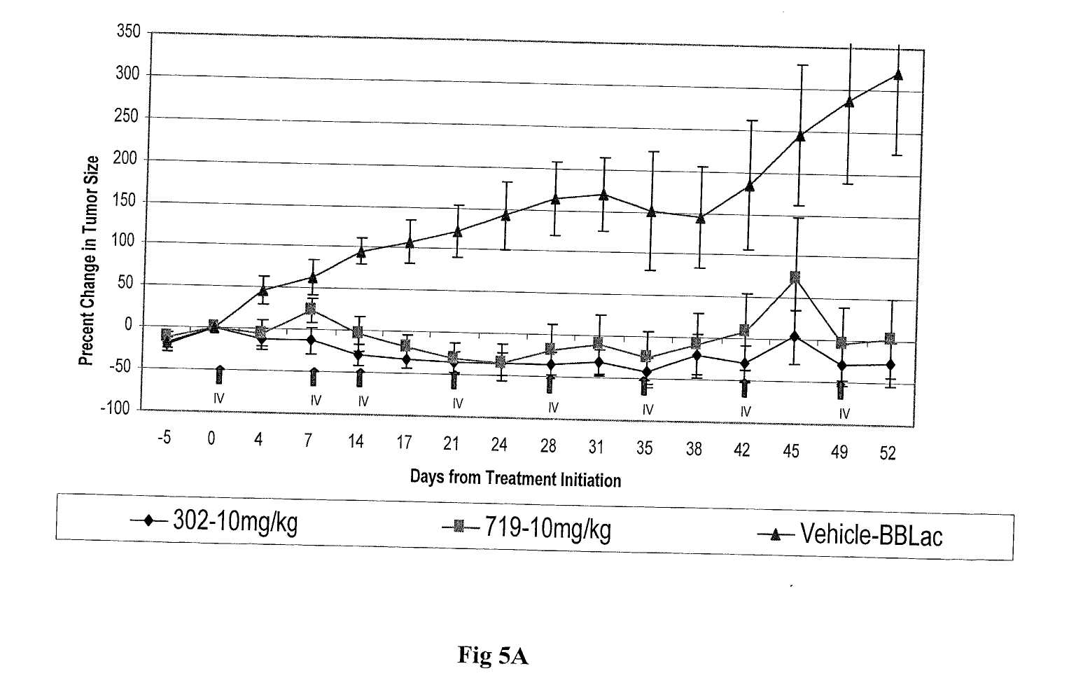

The results are shown in FIGS. 3 and 4. The data in these figures show that five different compounds, comprising five different Lyn derived peptides K055H101 (SEQ ID NO: 2); K055H123 (SEQ ID NO: 18); K055H137 (SEQ ID NO: 26); K055H302 (SEQ ID NO: 61); K055H719 (SEQ ID NO: 75), were able to inhibit growth of two different lines of prostate cancer cells PC-3 and DU-145.

### EXAMPLE 3

### Preparation of B-blac Formulation

15 mg of the compound were dissolved in 0.25 ml of 4% benzyl alcohol, 4% Pluronic L44 (BASF, Mount Olive, N.J.) and 2% benzyl benzoate in propylene glycol. To this, 0.125 ml of 2.2% glycine in DDW and 0.125 ml of 50 mM sodium bicarbonate were added while vigorously stirring the tube. The preparation was heated to 100° C. for 15 min., then homogenized with Polytron (Kinematica, Luzan, Switzerland) for 2′ during which 0.5 ml of 0.3 M lactose were gradually added.

The sequence of heating and homogenizing was repeated once again and after that the preparation was sterilized by heating to 100° C. for 30 min.

### EXAMPLE 4

### Prostate Cancer Tumor Shrinkage in Nude Mice

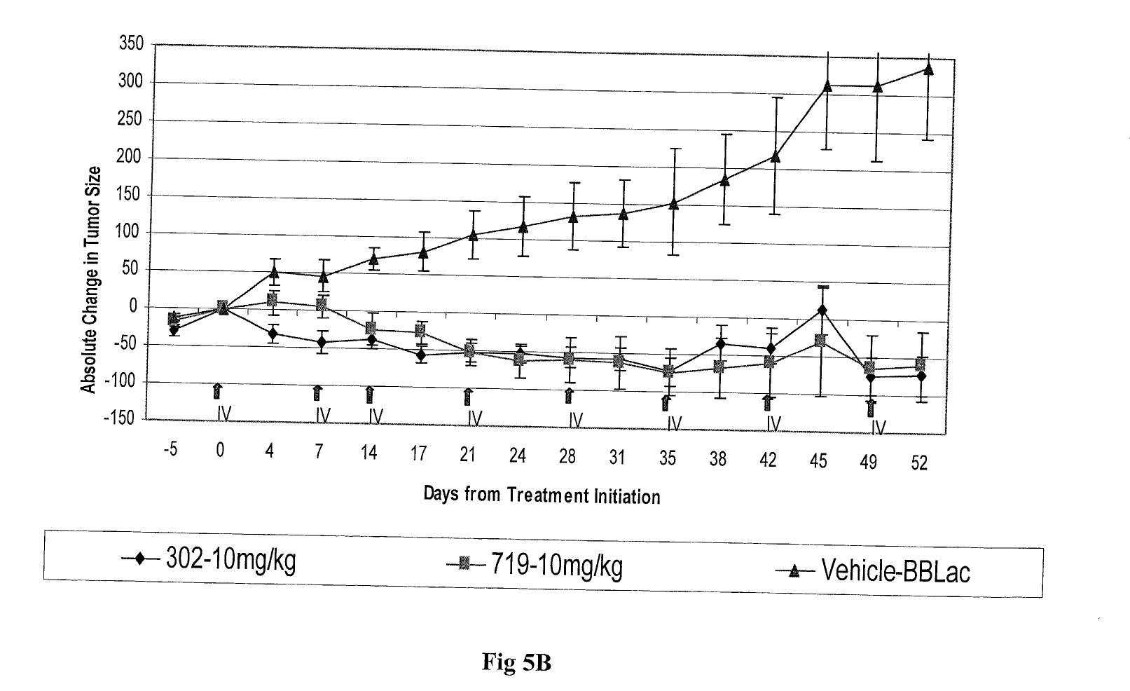

The hormnone-refractory human prostate cancer cell line, DU-145, was grown in RPMI-1640 culture medium with 10% fatal calf serum plus penicillin (100 U/ml), streptomycin (100 μg/ml), glutamine (2 mM) (see Example 2). The DU-145 cells were harvested and injected subcutaneously into male nude mice strain CDl of about 6-7 weeks of age, 5×10⁶ cells per mouse. After about 6 to 8 weeks, when the tumors became palpable, treatment of these mice was started by i.v. injection of 10 mg/kg of a solution comprising either compound K055H302 (SEQ ID NO: 61) or K055H719 (SEQ ID NO: 75). The compound solutions were prepared by taking BBlac formulation (see example 3) and diluting it 1:8 with lactose (0.3M). Mice received 0.2 ml of this solution. Control mice received i.v. injections of vehicle only. Tumor volume was measured twice a week. The results in FIG. 5A shows the change in tumor size, in percentage, from initial tumor, averaged for each group. The results in FIG. 5B show the absolute change in tumor size as compared to control averaged for each group.

As can be seen the tumor diminishes in size with time when compound injections are administered. By contrast, the tumors in control animals grow exponentially over the same time period. Clearly, the compound had a very significant effect on the decrease of the size prostate cancer. In some animals tumor was completely eliminated.

### EXAMPLE 5

### Change of Phosphorylation of Substrates.

Experimental: cell lymphocytes cell line WEHI-231 was used. 5×10⁶ WEHI-231 cells/sample were washed with serum-free RPMI media (cells were spun at 1700 rpm for 5 min. at 4° C.). The cells were suspended in serum-free RPMI media at 2×10⁷ cells/ml, and lysed by addition of an equal volume cold 2×LB (80 mM Tris pH 7.5, 2% NP-40, 1% DOC, 0.2 SDS, 50 mM NaPPi, 100 mM NaF, 2 mM Na₃VO₄, protease inhibitor mix) on ice for 15 min. The resulting mixture was spun for 20 min. 17,000 rpm at 4° C. and supernatantly the cell extract was saved.

Immuno-precipitation (IP) of each target-protein was done in one batch: to the cell extract 2 μg of appropriate Ab/reaction were added and then cells were rotated o/n on at 4° C. 30 μl 50% of slurry sample of protein A/G beads (prewashed 3 times with cold 1×LB) were again added for 3 hr at 4° C. The IP complex was washed (×2) with cold 1×LB and (×2) with cold reaction buffer (50 mM Tris pH 7.5, 10 mM MgCl₂, 0.1 mM Na₃VO₄, 1 mM DTT). The resulting mixture was spun for 1 min. 14,000 rpm at 4° C. and each IP batch was divided into separate tubes.

For the kinase assay: appropriate volumes of the compound of K055H302 (SEQ ID NO: 61) was added, or a control compound comprising an irrelevant sequence (obtained from a different kinase GRK) or a control of vehicle alone were added to each sample, and incubated for 20 min. at 30° C. Then 10 μM ATP and 5 units exogenous Lyn were added and incubated for 20 min. at 30° C. The reaction was stopped by addition of 8 μl 5×SDS sample buffer and boiled for 5 min. at 100° C. The resulting samples were separated on SDS-PAGE and blotted.

Western blot analysis was carried out with anti-phosphotyrosine, followed by stripping and rehybridization with the relevant antibodies.

The antibodies used in various assays:

| | |
|---|---|
| A-pTyr: | Upstate Biotechnology catalog #05-321 |
| A-CD19: | Pharmingen catalog #09651D |
| A-Lyn: | Santa Cruz sc-15 (44) |
| A-Syk: | Santa cruz sc-1077 (N-19) |
| A-Vav: | Santa Cruz sc-132 (C-14). |

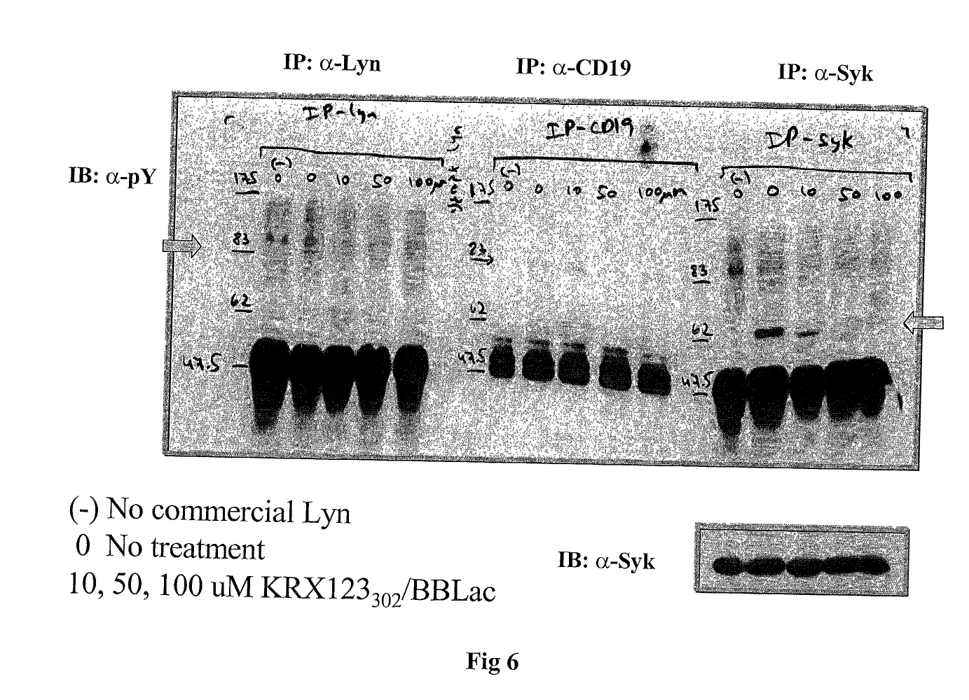

The results are shown in FIG. 6. These results show blots for three immunoprecipitates which are all substrates of Lyn: Lyn itself, CD19 and Syk and the level of phosphorylation is indicated in the absence of Lyn (0), and in the presence of Lyn (+) with increasing concentrations (0, 10, 50 and 100 μM) of the compound K055H302 (SEQ ID NO: 61) in B-blac(see example 3). Phosphorylation level was determined with anti-phosphotyrosine.

As can be seen Lyn, CD19 and Syk all showed dose-dependent decreased phosphorylation in the presence of the compound of the invention, thus indicating that the compound is a true LAST inhibitor, as evident by a decrease in the level of its phosphorylation, and that its effects in vivo and in vitro, shown in the above examples were through inhibition of LAST.

### EXAMPLE 6

### Soft Agar Assay

Reference: Hansen et al., J Immunol Met. 119:203 (1989).

Medium I: 2×RPMI Medium

20% Fetal Calf Serum (FCS)

4 mM Glutamine

Den/Strep

Medium II: dilution of Mediuml in tissue culture H₂O

Sea Plaque Agarose—FMC BioProducts Catal. #50102

Working solution: 2% agarose=2 grams /100 ml H₂O

0.6% agarose=0.6 grams/100 ml H₂O

MTT—sigma Catalog# M-2128

Working solution: 5 mg/ml PBS, store in dark at 4° C.

Solubilization Buffer

SDS Electrophoresis Grade—Fisher Catalog #BP166

N.N-Dimethyl-formamide (DMF) -Fisher Catolog # BP 1160

Acetic Acid, Glacial—fisher catalog #A38 Working solution:

Dissolve 40 g SDS in 70 ml warm H₂O and 100 ml DMF, stir in low heat

When SDS is almost solubilized, add 5 ml 80% acetic acid and 5 ml in HCL to solution. Adjust volume to 200 ml.

Procedure:

1. Melt agarose in a microwave mix 2% agarose 1:1 with Medium I to give 1% agarose in IX medium.

2. Dispense 100 μl into each well of a 96-well tissue culture plate.

3. Allow base layer to solidify at 40° C. for 15 minutes.

4. Mix 0.6% agarose 1:1 with Medium 1 containing DU-145 cells (4000 cells/well) in the presence or absence (control) of the compound of SEC ID 61 and plate 50 μl to each well on top of the under layer.

5. Allow the lower to solidify at 4° C. for 15 minutes.

6. Add 50 μl of 4×drug dilution in Medium II on top of the gel.

7. Incubate plate for 7 days at 37° C. 5% CO₂.

8. At end point, add 25 μl MTT to each well.

9. Incubate plate at 37° C., 5% CO₂ for 4 hours.

10. After 4 hours, add 100 μl solubilization solution to each well.

11. Let plat sit overnight in a sealed, humidified container to completely solubilized formazan crystals.

12. Read absorbance at 570 nm wavelength with a reference wavelength of 630 nm using a Dynatech ELISA plate reader, Model MR 500.

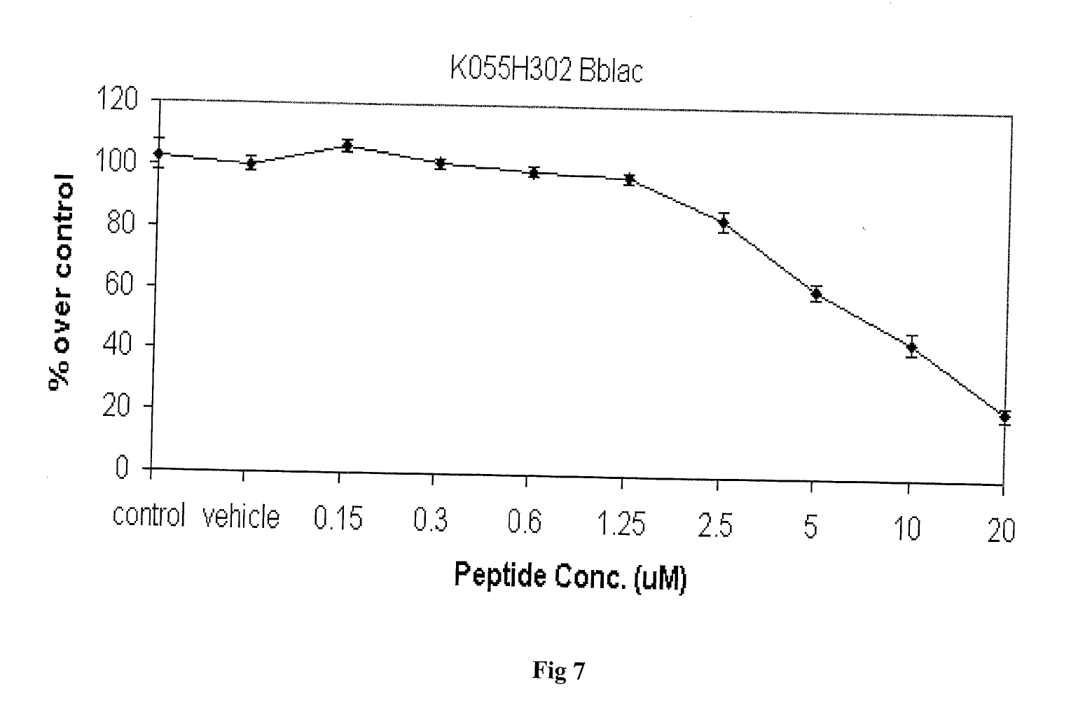

The results are shown in FIG. 7, which show the level of invasivness of DU-145 cells into the soft agar as compared to control in the presence of varying concentrations of the compound of the invention K055H302 (SEQ ID NO: 61). As can be seen a compound comprising an HJ-loop Lyn derived peptide, SEQ Id No 61 was able to reduce the invasiveness of prostate cancer cells DU-145 in a dose dependent manner indicating that the reduction of cancer growth can proceed not only by decrease of proliferation of the cells but also by decrease of their invasiveness.

### EXAMPLE 7

### Interruption Of Interaction Between Lyn And Its Substrate Syk In The Presence Of The Compound Of The Invention.

For proving that thr compound of the invention (SEQ ID NO 61) ,comprising a Lyn derived peptide, blocks the complexation of Lyn with its substrate Syk the amounts of Syk present together with Lyn-kinase, in the presence of varying concentrations of the compound was measured by co-immuno-precipitation (co-IP).

WEHI-231 cells were incubated with 10, 50 and 100 mM of the compound of SEQ ID NO: 61 for 2 hours and following stimulation with a-IgM

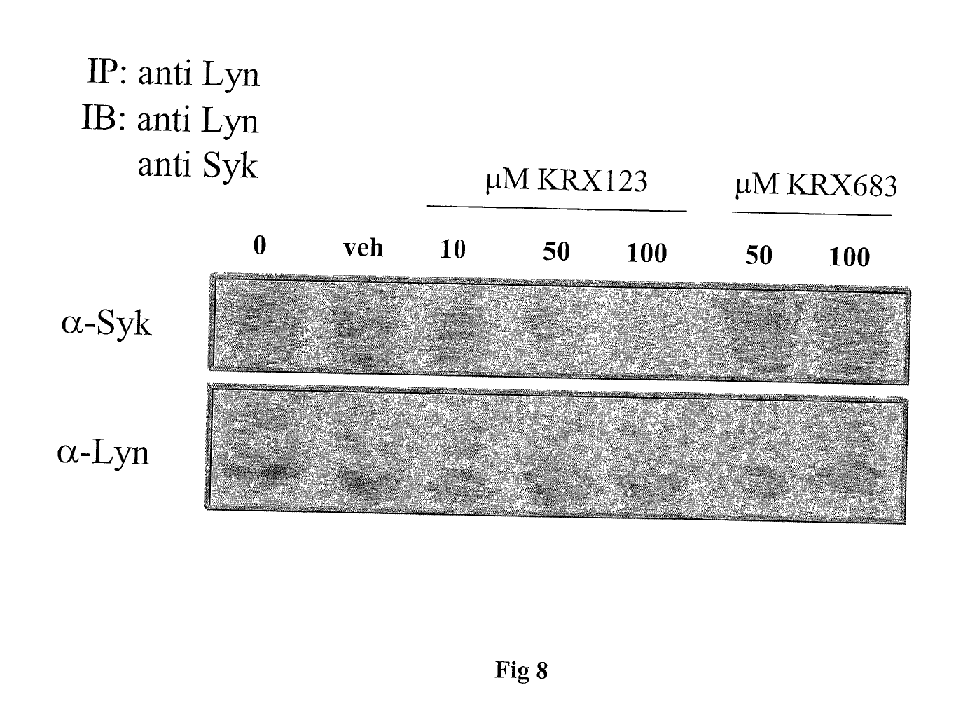

The Lyn was than immunoprecipitated using suitable abti-Lyn antibodies (see procedure of example 5). The Lyn-immunopercipitate was co-immunoreacted with anti-Syk abtibodies. The results are shown in FIG. 8. which demonstrates that Syk levels in the Lyn-immunoprecipitates decreased in a dose dependent manner (the amounts of the Lyn itself were not changes). These results support the theory of the invention that the compound comprising the Lyn-derived peptide interrupts the interaction of the Lyn kinase and its substrate (Syk) as can be seen by the decrease in the amount of Syk complexed with the Lyn-kinase. An irrelevant compound comprising a peptide derived from the HJ-loop region of another kinase (GRK) termed “683” showed no effect.

1. A method for the reduction of the growth of prostate cancer cells, the method comprising:
contacting the prostate cancer cells with an effective amount of a compound comprising a sequence selected from the group consisting of:
(a) a sequence which is a continuous stretch of at least five amino acids present in Lyn-kinase in positions 434-458 (HJ loop);
(b) a sequence which is a continuous stretch of at least five amino acids present in Lyn kinase in positions 318-336 (αD region);
(c) a sequence which is a continuous stretch of at least five amino acids present in Lyn-kinase in positions 305-316 (B4-B5 region);
(d) a sequence which is a continuous stretch of at least five amino acids present in Lyn kinase in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified ;and/or up to 20% of the amino acids have been deleted; provided that at least 50% of the amino acids in the parent sequences of (a) to (d) are maintained unaltered in the variant, and provided that the variant maintains the biological activity of the parent sequences of (a) to (d);
(f) a sequence of any one of (a) to (e) wherein at least one of the amino acids is replaced by the corresponding D- amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(i) a combination of two or more of the sequences of (a) to (h).

2. A method according to claim 1, wherein the compound comprises a sequence of (a)-( HJ-loop), (e), (f), (g) and (h).

3. A method according to claim 2, wherein the sequence of (a) is in positions 436 to 441 of the Lyn kinase.

4. A method according to claim 2, wherein the variant of (e) is produced by a combination of substitutions and chemical modifications.

5. The method of claim 1, wherein the compound is selected from the group consisting of K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74); SEQ ID NO: 75, and a compound comprising any one of the sequences of SEQ ID NOS: 76 to 81.

6. A method according to claim 1, wherein the compound comprises a moiety for transfer across cell membranes in association with the sequence of any one of (a) to (i).

7. A method according to claim 6, wherein the moiety is a hydrophobic moiety.

8. A method for the treatment of prostate cancer in an individual comprising administering to the individual a therapeutically effective amount of a compound comprising a sequence selected from the group consisting of:
(a) a sequence which is a continuous stretch of at least five amino acids present in Lyn-kinase in positions 434-458 (HJ loop);
(b) a sequence which is a continuous stretch of at least five amino acids present in Lyn kinase in positions 318-336 (αD region);
(c) a sequence which is a continuous stretch of at least five amino acids present in Lyn-kinase in positions 305-316 (B4-B5 region);
(d) a sequence which is a continuous stretch of at least five amino acids present in Lyn kinase in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequences of (a) to (d) are maintained unaltered in the variant, and provided that the variant maintains the biological activity of the parent sequences of (a) to (d);
(f) a sequence of any one of (a) to (e) wherein at least one of the amino acids is replaced by the corresponding D- amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(i) a combination of two or more of the sequences of (a) to (h).

9. A method according to claim 8, wherein the compound comprises a sequence of (a)-( HJ-loop), (e), (f), (g) and (h).

10. A method according to claim 9, wherein the sequence of (a) is in positions 436 to 441 of the Lyn kinase.

11. A method according to claim 9, wherein the variant of (e) is produced by a combination of substitutions and chemical modifications.

12. The method of claim 8, wherein the compound is selected from the group consisting of K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74); SEQ ID NO: 75, and a compound comprising any one of the sequences of SEQ ID NOS: 76 to81.

13. A method according to claim 8, wherein the compound comprises a moiety for transfer across cell membranes in association with the sequence of any one of (a) to (i).

14. A method according to claim 13, wherein the moiety is a hydrophobic moiety.

15. A method of reducing the growth of prostate cancer cells comprising contacting the cells with an inhibitor of Lyn-associated signal transduction (LAST), whereby said contact results in a reduction of growth of said prostate cancer cells.

16. The method of claim 15, wherein the LAST inhibitor is selected from the group consisting of:
(i) a compound comprising a sequence selected from the group consisting of:
(a) a sequence which is a continuous stretch of at least five amino acids present in Lyn-kinase in positions 434-458 (HJ loop);
(b) a sequence which is a continuous stretch of at least five amino acids present in Lyn kinase in positions 318-336 (αD region);
(c) a sequence which is a continuous stretch of at least five amino acids present in Lyn-kinase in positions 305-316 (B4-B5 region);
(d) a sequence which is a continuous stretch of at least five amino acids present in Lyn kinase in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequence of (a) to (d) are maintained unaltered in the variant, and provided that the variant maintains the biological activity of the parent sequence of (a) to (d);
(f) a sequence of any one of (a) to (e) wherein at least one of the amino acids is replaced by the corresponding D- amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(i) a combination of two or more of the sequences of (a) to (h);
(ii) an antibody, or antigen-binding portion thereof, reactive with Lyn-kinase or an immunogenic component thereof;
(iii) an antisense nucleic acid sequences complementary to a region in the Lyn-kinase gene or Lyn-kinase RNA, so that hybridization between said antisense and said gene or hybridization between said antisense and said RNA, results in decrease in expression of Lyn-kinase;
(iv) a ribozyme that specifically cleaves Lyn RNA
(v) an expression constructs coding for negative dominant Lyn-kinase and
(vi) small organic molecules.

17. The method of claim 16, wherein said small organic molecule is a pyrazolo pyrimidine-type inhibitor.

18. The method of claim 16, wherein said compound of (i) is selected from the group consisting of K055H007 (SEQ ID NO: 1); K055H101 (SEQ ID NO: 2 K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74), SEQ ID NO: 75, and a compound comprising of any one of SEQ ID NOS: 76 to.

19. A method of treatment of prostate cancer in a subject in need thereof, comprising: administering to said subject a therapeutically effective amount an inhibitor of Lyn-associated signal transduction (LAST), wherein said administration results in a reduction or stasis of said prostate cancer.

20. The method of claim 19, wherein the LAST inhibitor is selected from the group consisting of:
(vii) a compound comprising a sequence selected from the group consisting of:
a sequence which is a continuous stretch of at least five amino acids present in Lyn-kinase in positions 434-458 (HJ loop);
(k) a sequence which is a continuous stretch of at least five amino acids present in Lyn kinase in positions 318-336 (αD region);
(l) a sequence which is a continuous stretch of at least five amino acids present in Lyn-kinase in positions 305-316 (B4-B5 region);
(m) a sequence which is a continuous stretch of at least five amino acids present in Lyn kinase in positions 291-308 (A-region);
(n) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequence of (a) to (d) are maintained unaltered in the variant, and provided that the variant maintains the biological activity of the parent sequence of (a) to (d);
(o) a sequence of any one of (a) to (e) wherein at least one of the amino acids is replaced by the corresponding D- amino acid;
(p) a sequence of any one of (a) to (f) wherein at least one of the peptidic backbones has been altered to a non-naturally occurring peptidic backbone;
(q) a sequence being the sequence of any one of (a) to (g) in reverse order; and
(r) a combination of two or more of the sequences of (a) to (h);
(viii) an antibody, or antigen-binding portion thereof, reactive with Lyn-kinase or an immunogenic component thereof;
(ix) an antisense nucleic acid sequences complementary to a region in the Lyn-kinase gene or Lyn-kinase RNA, so that hybridization between said antisense and said gene or hybridization between said antisense and said RNA, results in decrease in expression of Lyn-kinase;
(x) a ribozyme that specifically cleaves Lyn RNA
(xi) an expression constructs coding for negative dominant Lyn-kinase; and
(xii) small organic molecules.

21. The method of claim 20, wherein said small organic molecule is a pyrazolo pyrimidine-type inhibitor.

22. The method of claim 20, wherein said compound of (i) is selected from the group consisting of K055H007 (SEQ ID NO: 1); K055H101 (SEQ ID NO: 2 K055H007 (SEQ ID NO: 1); K055H101 (SEQ ID NO: 2); K055H104 (SEQ ID NO: 3);K055H108 (SEQ ID NO: 4);K055H110 (SEQ ID NO: 5);K055H111 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74), SEQ ID NO: 75, and a compound comprising of any one of SEQ ID NOS: 76 to.

23. A compound selected from the group consisting of: K055H007 (SEQ ID NO: 1); K055H101 (SEQ ID NO: 2 K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); and K055H925 (SEQ ID NO: 74) and K055H719 SEQ ID NO: 75, and a sequence of any one of SEQ ID NOS: 76-81 associated with a moiety for transfer across membranes.

24. A pharmaceutical composition for treating prostate cancer, comprising a pharmaceutically acceptable carrier and a compound comprising a sequence selected from the group consisting of:
(a) a sequence which is a continuous stretch of at least five amino acids present in Lyn-kinase in positions 434-458 (HJ loop);
(b) a sequence which is a continuous stretch of at least five amino acids present in Lyn kinase in positions 318-336 (αD region);
(c) a sequence which is a continuous stretch of at least five amino acids present in Lyn-kinase in positions 305-316 (B4-B5 region);
(d) a sequence which is a continuous stretch of at least five amino acids present in Lyn kinase in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified ;and/or up to 20% of the amino acids have been deleted; provided that at least 50% of the amino acids in the parent sequence of (a) to (d) are maintained unaltered in the variant, and provided that the variant maintains the biological activity of the parent sequence of (a) to (d);
(f) a sequence of any one of (a) to (e) wherein at least one of the amino acids is replaced by the corresponding D- amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(i) a combination of two or more of the sequences of (a) to (h);

25. The pharmaceutical composition according to claim 24, wherein the compound comprises a sequence of (a-)( HJ-loop), (e), (f), (g) and (h).

26. The pharmaceutical composition according to claim 25, wherein the sequence of (a) is in positions 436 to 441 of the Lyn kinase.

27. The pharmaceutical composition according to claim 24, wherein the compound is selected from the group consisting of K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H113 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74) SEQ ID NO: 75, and a compound comprising any one of SEQ ID NOS: 76 to 81.

28. The pharmaceutical composition according to claim 24, wherein the compound comprises a moiety for transfer across cell membranes in association with the sequence of any one of (a) to (i).

29. The pharmaceutical composition according to claim 28, wherein the moiety is a hydrophobic moiety.

30. The pharmaceutical composition for treating prostate cancer, comprising a pharmaceutically acceptable carrier and at least one inhibitor of Lyn-associated signal transduction (LAST) for the preparation of a medicament.

31. The pharmaceutical composition according to claim 30, wherein the LAST inhibitor is selected from the group consisting of:
(i) a compound comprising a sequence selected from the group consisting of:
(a) a sequence which is a continuous stretch of at least five amino acids present in Lyn-kinase in positions 434-458 (HJ loop);
(b) a sequence which is a continuous stretch of at least five amino acids present in Lyn kinase in positions 318-336 (αD region);
(c) a sequence which is a continuous stretch of at least five amino acids present in Lyn-kinase in positions 305-316 (B4-B5 region);
(d) a sequence which is a continuous stretch of at least five amino acids present in Lyn kinase in positions 291-308 (A-region);
(e) a variant of a sequence according to any one of (a) to (d) wherein up to 40% of the amino acid of the native sequence have been replaced with a naturally or non-naturally occurring amino acid or with a peptidomimetic organic moiety; and/or up to 40% of the amino acids have their side chains chemically modified; and/or up to 20% of the amino acids have been deleted; provided that at least 50% of the amino acids in the parent sequence of (a) to (d) are maintained unaltered in the variant, and provided that the variant maintains the biological activity of the parent sequence of (a) to (d);
(f) a sequence of any one of (a) to (e) wherein at least one of the amino acids is replaced by the corresponding D- amino acid;
(g) a sequence of any one of (a) to (f) wherein at least one of the peptidic backbones has been altered to a non-naturally occurring peptidic backbone;
(h) a sequence being the sequence of any one of (a) to (g) in reverse order; and
(i) a combination of two or more of the sequences of (a) to (h);
(ii) an antibody, or antigen-binding portion thereof, reactive with Lyn-kinase or an immunogenic component thereof;
(iii) an antisense nucleic acid sequences complementary to a region in the Lyn-kinase gene or Lyn-kinase RNA, so that hybridization between said antisense of said gene, or hybridization between said antisense and said RNA results in decrease in expression of Lyn-kinase;
(iv) a ribozyme that specifically cleaves Lyn RNA
(v) expression constructs coding for negative dominant Lyn-kinase; and
(vi) small organic molecules.

32. The pharmaceutical composition of claim 31, wherein said small organic molecule is a pyrazolo pyrimidine-type inhibitor.

33. The pharmaceutical composition of claim 31, wherein said compound of (i) is selected from the group consisting of K055H007 (SEQ ID NO: 1); K055H101 (SEQ ID NO: 2 K055H007 (SEQ ID NO: 1); K055H101 (SEQ ID NO: 2); K055H104 (SEQ ID NO: 3); K055H108 (SEQ ID NO: 4); K055H110 (SEQ ID NO: 5); K055H111 (SEQ ID NO: 6); K055H112 (SEQ ID NO: 7); K055H1 13 (SEQ ID NO: 8); K055H114 (SEQ ID NO: 9); K055H115 (SEQ ID NO: 10); K055H116 (SEQ ID NO: 11); K055H117 (SEQ ID NO: 12); K055H118 (SEQ ID NO: 13); K055H119 (SEQ ID NO: 14); K055H120 (SEQ ID NO: 15); K055H121 (SEQ ID NO: 16); K055H122 (SEQ ID NO: 17); K055H123 (SEQ ID NO: 18); K055H124 (SEQ ID NO: 19); K055H125 (SEQ ID NO: 20); K055H129 (SEQ ID NO: 21); K055H130 (SEQ ID NO: 22); K055H134 (SEQ ID NO: 23); K055H135 (SEQ ID NO: 24); K055H136 (SEQ ID NO: 25); K055H137 (SEQ ID NO: 26); K055H138 (SEQ ID NO: 27); K055H139 (SEQ ID NO: 28); K055H140 (SEQ ID NO: 29); K055H142 (SEQ ID NO: 30); K055H143 (SEQ ID NO: 31); K055H144 (SEQ ID NO: 32); K055H145 (SEQ ID NO: 33); K055H146 (SEQ ID NO: 34); K055H147 (SEQ ID NO: 35); K055H148 (SEQ ID NO: 36); K055H149 (SEQ ID NO: 37); K055H152 (SEQ ID NO: 38); K055H153 (SEQ ID NO: 39); K055H154 (SEQ ID NO: 40); K055H155 (SEQ ID NO: 41); K055H161 (SEQ ID NO: 42); K055H162 (SEQ ID NO: 43); K055H163 (SEQ ID NO: 44); K055H164 (SEQ ID NO: 45); K055H165 (SEQ ID NO: 46); K055H166 (SEQ ID NO: 47); K055H167 (SEQ ID NO: 48); K055H168 (SEQ ID NO: 49); K055H169 (SEQ ID NO: 50); K055H170 (SEQ ID NO: 51); K055H171 (SEQ ID NO: 52); K055H172 (SEQ ID NO: 53); K055H173 (SEQ ID NO: 54); K055H174 (SEQ ID NO: 55); K055H175 (SEQ ID NO: 56); K055H176 (SEQ ID NO: 57); K055H177 (SEQ ID NO: 58); K055H300 (SEQ ID NO: 59); K055H301 (SEQ ID NO: 60); K055H302 (SEQ ID NO: 61); K055H304 (SEQ ID NO: 62); K055H305 (SEQ ID NO: 63); K055H306 (SEQ ID NO: 64); K055H307 (SEQ ID NO: 65); K055H801 (SEQ ID NO: 66); K055H902 (SEQ ID NO: 67); K055H908 (SEQ ID NO: 68); K055H910 (SEQ ID NO: 69); K055H911 (SEQ ID NO: 70); K055H912 (SEQ ID NO: 71); K055H919 (SEQ ID NO: 72); K055H923 (SEQ ID NO: 73); K055H925 (SEQ ID NO: 74), SEQ ID NO: 75, and a compound comprising any one of SEQ ID NOS: 76 to 81.
